SEQUENCE LISTING (12 sequences)

SEQ ID NO: 1 - DNA, 17 bp, Artificial Sequence
  primer containing synthetic TATA box
agcttgagggtataatg

SEQ ID NO: 2 - DNA, 17 bp, Artificial Sequence
  primer containing synthetic TATA box
actcccatattactcga

SEQ ID NO: 3 - DNA, 36 bp, Artificial Sequence
  PCR primer for amplification of Drosophila melanogaster DNA
gatccgagacaagggttcaatgcacttgtccaatga

SEQ ID NO: 4 - DNA, 36 bp, Artificial Sequence
  PCR primer for amplification of Drosophila melanogaster DNA
gctctgttcccaagttacgtgaacaggttactctag

SEQ ID NO: 5 - DNA, 147 bp, Artificial Sequence
  multimerized binding site of Drosophila melanogaster hsp27 gene
gatccgagacaagggttcaatgcacttgtccaatgagatccgagacaagggttcaatgcacttgtccaatgagatctcattggacaagtgcattgaaccttgtctcggatctcattggacaagtgcattgaacccttgtctcggatc

SEQ ID NO: 6 - DNA, 2840 bp, Manduca sexta
  CDS (361)..(2031): Manduca sexta Ecdysone Receptor
tccgttgacgacggtcgcacgcgtgcaacgtgctcgtttttacggctcaagcgaacgcgtaacctccgtctccacatcaccgagcgaactctagaactcgcgtactcttctcacctgttgcttcggattgtgttgtgactgaaaagcgacgcgtatcgtggtcgaagattctctataagtgcataatatattcgagacagtggatagcgattcgtttcggtttcatcgcgcggatgagtggttcatgcccgtagagacgcgtttagatagttatggcgaggaaaaagtgaagtgaaagcctacgtcagaggatgtccctcggtggtcacggaagccggggcgtgtgacgcgctcttcgacatgagacgccgctggtcaaacaacggatgtttccctctgcgaatgtttgaggagagctcctctgaagtgacttcttcctcggcgttcgggatgccggcggccatggtaatgtcaccggagtcgctggcgtcgccagagtacggcggcctcgagctctggagctacgatgagaccatgacaaactatccggcgcagtcactgctcggcgcgtgtaatgcgccgcagcagcagcagcaacagcaacaacagcagccgtccgctcagccgctgccgtctatgccgctgccgatgcctcctacaactcctaaatcagagaacgagtccatgtcgtcaggtcgagaagaattatcaccggcctcaagtataaatggatgtagtactgatggggaaccaagacgacagaagaaagggccagcgccgcgccagcaggaggaactgtgccttgtttgcggcgacagggcttcgggatatcactataacgcgcttacgtgcgaaggatgtaaagggttcttcaggcggagtgtgaccaagaatgcggtatatatttgtaaatttggacacgcctgcgagatggacatgtacatgaggagaaaatgccaagagtgtcggttgaagaaatgcctcgcggtgggcatgaggcccgagtgcgtcgtcccagagtccacgtgcaagaacaaaagaagagaaaaggaagcacagagagaaaaagacaaactgccagtcagtacgacgacagtggacgatcatatgcctgccataatgcaatgtgaccctccgcccccagaggcggcaaggattcacgaagtggtcccgaggttcctaacggagaagctaatggagcagaacagactgaagaatgtgacgccgctgtcggcgaaccagaagtccctgatcgcgaggctcgtgtggtaccaggaggggtacgagcagccgtcggaggaagatctcaagagagttacacagacatggcagttagaagaagaagaagaggaggaaactgacatgcccttccgtcagatcacagagatgacgatcttaacagtgcagcttattgtagaattcgcaaagggactaccgggattctccaagatatctcagtccgatcaaattacattattaaaggcgtcatcaagcgaagtgatgatgctgcgagtggcgcgacggtacgacgcggcgacggacagcgtgctgttcgcgaacaaccaggcgtacacgcgcgacaactaccgcaaggcgggcatgtcctacgtcatcgaggacctgctgcacttctgtcggtgtatgtactccatgagcatggacaatgtgcactacgcgctgctcaccgccatcgttatattctcagaccggccaggcctcgagcaaccccttttagtggaggaaatccagagatactacttgaagacgctgcgggtttacattttaaatcagcacagcgcgtcgcctcgctgcgccgtgctgttcggcaagatcctcggcgtgctgacggaactgcgcacgctcggcacgcagaactccaacatgtgcatctcgctgaagctgaagaacaggaaacttccgccattcctcgaggagatctgggacgtggccgaagtgtcgacgacgcagccgacgccgggggtggcggcgcaggtgacccccatcgtggtggacaaccccgcggcgctctagctggcgcgccggcgccgcgccccgccgcccccgccgccgccgctcccccgcgccgccgccgcgcgcccccgcggcctgcgctgagtgcgggacccgccccgaggagagaacgctcatagactggctagttttagtgaagtgcacggacgcgatcgtgggaccgcatcgacgcgtccgtgaggacagtgcaaatattaccgctagggccggttcgtacgtgtccggtgaccgacgacgatgatgcgcgtgagattagtgaatatatgtgttgttgaacgtttggagagtatatttagtgttgatcgtcgggagcgcgcggccggcgcgtgtcggcgagctgtccgccgcgcgccggccgcggcgactccgcgtttttttcgtttgcgaccggaaaccgagtcggtcactcggatacgcccgtatgataagacttctttcgataaataagttcacctgtattgcgcgtacatacgagaattataaagaaaaaaagtaatatatgaagagatgtttctattgggtgaaaagtttaaacttatgtttatttaccaaaattaactatacgttgatcgaccttttgactataatattgtgctgggtcgttggcagcggccgacgaacgcgcgccgaccatatttgtttatatatagtttatgtgagacgttatcgtgtcgtgtccacttagttccgattcatgttccaccaggtcggtgtagtgatcagggcgggccagggtgacggccaccacggataacaggcaaagagcgacgaatgttttcatgttgagactttgggagacgttattcctc

SEQ ID NO: 7 - protein, 556 aa, Manduca sexta
MRRRWSNNGCFPLRMFEESSSEVTSSSAFGMPAAMVMSPESLASPEYGGLELWSYDETMTNYPAQSLLGACNAPQQQQQQQQQQPSAQPLPSMPLPMPPTTPKSENESMSSGREELSPASSINGCSTDGEPRRQKKGPAPRQQEELCLVCGDRASGYHYNALTCEGCKGFFRRSVTKNAVYICKFGHACEMDMYMRRKCQECRLKKCLAVGMRPECVVPESTCKNKRREKEAQREKDKLPVSTTTVDDHMPAIMQCDPPPPEAARIHEVVPRFLTEKLMEQNRLKNVTPLSANQKSLIARLVWYQEGYEQPSEEDLKRVTQTWQLEEEEEEETDMPFRQITEMTILTVQLIVEFAKGLPGFSKISQSDQITLLKASSSEVMMLRVARRYDAATDSVLFANNQAYTRDNYRKAGMSYVIEDLLHFCRCMYSMSMDNVHYALLTAIVIFSDRPGLEQPLLVEEIQRYYLKTLRVYILNQHSASPRCAVLFGKILGVLTELRTLGTQNSNMCISLKLKNRKLPPFLEEIWDVAEVSTTQPTPGVAAQVTPIVVDNPAAL

SEQ ID NO: 8 - DNA, 30 bp, Artificial Sequence
  PCR primer for amplification of Manduca sexta DNA
ggatcctaaagcttcgtcgtcgacacttcg

SEQ ID NO: 9 - DNA, 30 bp, Artificial Sequence
  PCR primer for amplification of Manduca sexta DNA
ggatccatgggycgagaagaattrtcaccr

SEQ ID NO: 10 - DNA, 21 bp, Artificial Sequence
  PCR primer for amplification of Manduca sexta DNA
ccacrtcccagatctcctcga

SEQ ID NO: 11 - DNA, 19 bp, Artificial Sequence
  PCR primer for amplification of Manduca sexta DNA
aagcttgcccccccgaccg

SEQ ID NO: 12 - DNA, 25 bp, Artificial Sequence
  PCR primer for amplification of Manduca sexta DNA
tctagaggatcctacccaccgtact

(19) United States
(12) Patent Application Publication
(10) Pub. No.: US 2003/0109705 A1
(43) Pub. Date: Jun. 12, 2003

(54) Novel cyanoenamines useful as ligands for modulating gene expression in plants or animals

(76) Inventor: Jonas Grina, Superior, CO (US)

Correspondence Address: JENKINS & WILSON, PA, 3100 TOWER BLVD, SUITE 1400, DURHAM, NC, 27707, US

(21) Appl. No.: 10/083,842

(22) Filed: Feb. 27, 2002

Related U.S. Application Data
(60) Provisional application No. 60/272,905, filed on Mar. 2, 2001.

(51) Int. Cl. C07D 471/02; C07D 285/12; C07D 285/14

(52) U.S. Cl. 544/242; 544/336; 546/122; 546/146; 546/174; 546/314; 548/136; 548/200; 548/206; 548/236; 548/318.5; 558/390

(57) ABSTRACT

Accordingly, the present invention provides a compound of Formula I, or tautomers or isomers thereof,

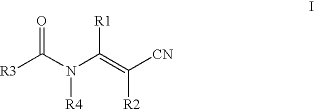

wherein: R1 is a branched chain lower alkyl (C3 to C8), cycloalkyl (C3 to C8), alkyl-substituted alkyl (C4 to C8), bicycloalkyl, 1-adamantyl, polyhaloalkyl, trialkylsilyl, unsubstituted phenyl or substituted phenyl; R2 and R3 are independently unsubstituted or substituted aromatic rings; and R4 is hydrogen, alkyl, alkylthio, alkylthioalkyl, acyloxyalkyl, alkyl, acyl, trialkylsilyl, or cyclized together with R3 and O to form a lactone of the tautomeric form.

### CROSS REFERENCE TO RELATED APPLICATIONS

The present patent application claims benefit of U.S. Provisional Patent Application Serial No. 60/272,905, filed Mar. 2, 2001 and is incorporated herein by reference.

### TECHNICAL FIELD

The present invention relates, in general, to novel compounds that are useful as ligands for modulating gene expression in living organisms (plants and/or animals). More particularly, the present invention relates to compounds that are cyanoenamines that are useful as non-steroidal ligands for modulating exogenous gene expression in eukaryotic organisms (i.e., those where the cell has a nucleus), more particularly plants, especially chlorophyll-containing plants.

Table of Abbreviations

| | |
|---|---|
| n-BuLi | n-butyllithium |
| t-butyl | tert-butyl |
| C | centigrade |
| DMAP | N,N-dimethylaminopyridine |
| DNA | deoxyribonucleic acid |
| EcR | ecdysone receptor |
| EC80 | effective concentration that produces |
| | an 80% effect |
| GC | gas chromatography |
| GR | glucocorticoid receptor |
| g | gram |
| Hv | Heliothis virescens |
| h | hour |
| L1 | first larval instar, namely the stage |
| | between the egg and the first molt |
| LC | liquid crystal |
| LDA | lithium diisopropylamide |
| MS | mass spectroscopy |
| mp | melting point |
| μM | micromole |
| mL | milliliter |
| mm | millimeter |
| min | minute |
| M | mole |
| NMR | nuclear magnetic resonance |
| # | number |
| ppm | parts per million |
| RNA | ribonucleic acid |
| RXR | retinoid X receptor |
| SPODLI | Spodoptera littoralis |
| THF | tetrahydrofuran |
| USP | ultraspiracle |

### BACKGROUND OF THE INVENTION

Precise temporal control of gene expression is a valuable tool in the field of genetic engineering. The ability to activate (i.e., to induce) or to suppress a gene is of vast importance in manipulating, controlling, and/or studying development and other physiological processes. Inducability is often valuable for foreign protein production, such as production of therapeutic proteins, industrial enzymes, and polymers, in both plants and animals.

Specifically in the case of plants, often desirable is the control of the timing and level of expression of a phenotypic trait in a plant, plant cell or plant tissue. Ideally, regulation of expression of such a trait can be achieved whenever desired by triggering gene expression with a chemical that is easily applied to field crops, ornamental shrubs and other plants of economic importance. This triggering mechanism for gene expression control is referred to as a gene switch. In order to avoid unexpected activation of the gene switch, the chemical should be one that is normally absent from the plant.

One such gene switch mechanism is the ecdysone receptor (EcR). EcR is a member of the nuclear hormone family of receptors. Members of this receptor family are multi-domain proteins, capable of regulating gene expression in response to a chemical ligand. The DNA binding domain (also known as the C domain) binds to a specific target DNA sequence. This specificity determines which target genes are activated by the receptor. The ligand binding domain (E domain) plays a critical role in the determination of ligand specificity as well as the ligand regulated activation property of the receptor. The hinge domain (domain D) resides between the DNA binding and ligand binding domains. The hinge domain modulates the receptor's response to ligand induction.

Ligands that are complementary to the ligand binding domain of the ecdysone receptor are known. Steroidal agonists such as 20-hydroxy ecdysone, muristerone, and ponasterone are capable of activating an ecdysone receptor gene switch. Non-steroidal agonists have advantages over steroidal agonists due to such factors as greater stability, cheaper cost, and environmental acceptance. One known non-steroidal agonist is the insecticide Tebufenozide (also known as the insecticide sold under the trademark MIMIC®).

Of interest is European Published Patent Application No. 0 965 644 A2 to Carlson et al., assignors to Rohm and Haas Company, which relates to a method of modulating exogenous gene expression in which an ecdysone receptor complex is contacted with a DNA construct having an exogenous gene under the control of a response element, and where the binding of the ecdysone receptor to the response element results in activation or suppression of the gene. The ligand is chosen from certain dibenzoyl-tert-butyl-hydrazine compounds.

As referred to herein, an “ecdysone receptor gene switch” means a gene switch comprising an ecdysone receptor. The ecdysone receptor gene switch may be a heterodimer of EcR and USP, or EcR and RXR. The heterodimerization partner may be native to the organism or cell type in which the gene switch is present, or the heterodimerization partner may be provided exogenously. The ecdysone receptor gene switch may be comprised only of EcR in the absence of a heterodimerization partner. EcR may be in its native form, as isolated from insects, comprising a DNA binding domain, hinge and ligand binding domain from an insect EcR. EcR may be a chimeric protein comprising a DNA binding domain from another EcR or another transcription factor such as Ga14. EcR may comprise its native activation domain or an activation domain of another protein. Furthermore, EcR may comprise a ligand binding domain of an insect ecdysone receptor or a ligand binding domain from a member of the nuclear hormone family of receptors.

Also of interest is International Publication No. WO 00/15791 to Albertsen et al., assignors to Pioneer Hi-Bred International, Inc. This Publication relates to novel ecdysone receptors from the insect species Ostrinia and the genus Pyrilidae and their use for gene regulation in plants.

Additionally of interest is International Publication No. WO 99/02683 to Gage et al., assignors to The Salk Institute for Biological Studies. This Publication relates to nuclear receptor proteins from the silk moth Bombyx mori, useful for the regulation of gene expression.

Also of interest is International Publication No. WO 96/37609 to Jepson et al., assignors to Zeneca, relating to the use of a chimeric ecdysone receptor gene switch in plants.

Of general background interest is each of the following describing examples of ecdysone receptor gene switches: No et al., Proc. Nat'l. Acad. Sci., 93: 3346-3351 (1996), describing EcR in mammalian cells; Godowski et al., International Publication No. WO 93/03162, describing EcR and chimeric EcR proteins and related gene switches; Evans et al., International Publication Nos. WO 99/58155 and WO 97/38117, describing EcR and chimeric EcR proteins and related gene switches; Martinez et al., Insect. Biochem. Mol. Biol., 29 (10):915-930 (October, 1999), describing a chimeric EcR gene switch in plants; Martinez et al., Plant J., 19(1):97-106 (July, 1999), describing a chimeric EcR gene switch in plants; Martinez et al., Mol. Gen. Genet., 261(3):546-552 (April, 1999), describing a chimeric EcR gene switch in plants; Suhret al., Proc. Nat'l. Acad. Sci. U.S.A., 95(14):7999-8004 (Jul. 7, 1998), describing a chimeric EcR switch in mammalian cells; and Hoppe et al., Mol. Ther., 1(2):159-164 (February, 2000), describing an adenovirus mediated EcR gene switch.

Especially of interest is U.S. Pat. No. 5,880,333 to Goff et al., assignors to Novartis Finance Corporation. This patent discloses a method of controlling gene expression in plants. Specifically, the method involves obtaining a transgenic plant that has at least 2 receptor expression cassettes and at least 1 target expression cassette. A first of the 2 receptor expression cassettes has a nucleotide sequence for a 5′ regulatory region operably linked to a nucleotide sequence that encodes a first receptor polypeptide and a 3′ termination region. A second of the 2 receptor expression cassettes has a nucleotide sequence for a 5′ regulator region operably linked to a nucleotide sequence that encodes a second receptor polypeptide and a 3′ termination region. The target expression cassette has a nucleotide sequence operably linked to a nucleotide sequence that encodes a target polypeptide and a 3′ termination region, wherein the 5′ regulatory region of the target expression cassette is activated by the first and second receptor polypeptides in the presence of a certain chemical ligand that is complimentary to the ligand binding domain of the receptor polypeptides, as a result of which expression of the target polypeptide is accomplished. In a preferred embodiment, the method involves expressing in a plant an insect EcR and a second receptor as a heterodimerization partner and activating the expression of a target polypeptide by contacting the plant cells with a ligand that is complimentary to the ligand binding domain of one of the receptors. The method of U.S. Pat. No. 5,880,333 to Goff et al. is useful for controlling various traits of agronomic importance, such as plant fertility.

Lastly, of interest is U.S. Provisional Application No. 60/242,969, filed Oct. 24, 2000, describing novel ecdysone receptor gene switches and methods of use, the disclosure of which is incorporated in its entirety.

All of the patents and published patent applications mentioned here are incorporated by reference.

Despite the plethora of available ecdysone receptor gene switch systems, there still remain a continuing need to develop non-steroidal ligands with increased activity as compared to known ligands and a need to develop ligands that demonstrate improved consistent activity in intact plants and animals.

### SUMMARY AND OBJECTS OF THE INVENTION

Accordingly, the present invention provides a compound comprising Formula I

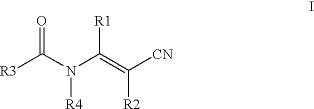

and also, Formula I may be in its tautomeric form comprising Formula II

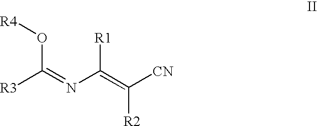

and also, Formula I may be in its isomeric form comprising Formula III

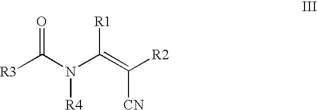

wherein:

R1

is a branched chain lower alkyl (C3 to C8), cycloalkyl (C3 to C8), alkyl-substituted alkyl (C4 to C8), bicycloalkyl, 1-adamantyl, polyhaloalkyl, trialkylsilyl, unsubstituted phenyl or optionally substituted phenyl;

R2 and R3

are independently unsubstituted or substituted aromatic rings, chosen from phenyl, pyridyl, pyrimidinyl, furyl, thiophenyl, pyrazinyl, pyrrolyl, pyrazolyl, 1,2,4-triazolyl, naphthyl, fluorenonyl, xanthenyl, 4-oxo-1,4-dihydro-(1,8)naphthyridinyl, thiazolyl, isothiazolyl, 1,3,4-thiadiazolyl, benzo-1,2,3-thiadiazolyl, oxazolyl, imidazolyl, quinolinyl, or isoquinolinyl, where a substituent on the rings is one or more chosen independently from hydrogen, alkyl (C1 to C4), alkoxy, alkoxyalkyl, hydroxy, amino, alkylamino, dialkylamino, acylamino, halo, haloalkyl, hydroxyalkyl, dihydroxyalkyl, alkoxycarbonyl, alkylaminocarbonyl, dialkylaminocarbonyl, unsubstituted or substituted alkylphenyl, unsubstituted or substituted phenyl, unsubstituted or substituted phenoxy, nitro, cyano, alkylthio, alkylsulfonyl, aminoalkyl, carboxyalkyl, or sulfonylalkyl; and

R4

is hydrogen, alkylthio, alkylthioalkyl, alkyloxyalkyl, acyloxyalkyl, alkyl, acyl, trialkylsilyl, or cyclized together with R3 and the O in Formula II to form a lactone.

The compounds described in the above paragraph are useful for modulation of an exogenous gene in a living organism. The compounds are also useful for the control of pests, such as anthropods, parasites, and the like, by acting as agonists of 20-hydroxyecdysone, the molting hormone.

Therefore, it is an object of the present invention to provide a compound that has the ability to activate or to suppress an exogenous gene.

It is another object of the present invention to provide a compound that is useful as a pesticide.

Some of the objects of the invention having been stated, other objects will become evident as the description proceeds, when taken in connection with the laboratory examples described below.

### DETAILED DESCRIPTION OF THE INVENTION

The inventive ligand is an addition to the ligands described in the above-noted U.S. Pat. No. 5,880,333 to Goff et. al.

A ligand according to the present invention is described by the below recited general Formula I and its below recited tautomer, Formula II, and its below recited isomer, Formula III:

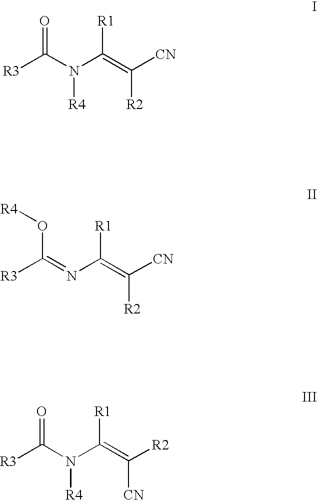

wherein:

R1

is a branched chain lower alkyl (C3 to C8), cycloalkyl (C3 to C8), alkyl-substituted alkyl (C4 to C8), bicycloalkyl, 1-adamantyl, polyhaloalkyl, trialkylsilyl, or optionally substituted phenyl;

R2 and R3

are independently optionally substituted aromatic rings, such as phenyl, pyridyl, pyrimidinyl, furyl, thiophenyl, pyrazinyl, pyrrolyl, pyrazolyl, 1,2,4-triazolyl, naphthyl, fluorenonyl, xanthenyl, 4-oxo-1,4-dihydro-(1,8)naphthyridinyl, thiazolyl, isothiazolyl, 1,3,4-thiadiazolyl, benzo-1,2,3-thiadiazolyl, oxazolyl, imidazolyl, quinolinyl, or isoquinolinyl. Substituents on these rings can be one or more chosen independently from hydrogen, alkyl (C1 to C4), alkoxy, alkoxyalkyl, hydroxy, amino, alkylamino, dialkylamino, acylamino, halo, haloalkyl, hydroxyalkyl, dihydroxyalkyl, alkoxycarbonyl, alkylaminocarbonyl, dialkyl-aminocarbonyl, (optionally substituted) alkylphenyl, (optionally substituted) phenyl, (optionally substituted) phenoxy, nitro, cyano, alkylthio, alkylsulfonyl, aminoalkyl, carboxyalkyl, and sulfonylalkyl; and

R4

is hydrogen, or a substituent that may be easily removed in planta, serving as an aid in absorption and/or translocation, such as alkylthio, alkylthioalkyl, alkyloxyalkyl, acyloxyalkyl, alkyl, acyl, trialkylsilyl, or cyclized together with R3 and the O in Formula II to form a lactone.

Halo may be selected from the group consisting of fluoro, chloro, bromo, iodo, and combinations thereof. The substituents on R2 and R3 may also be joined to form cyclic structures on adjacent atoms of the aromatic ring, such as 1,2-methylenedioxy and 1,2-difluoromethylenedioxy. The preferred R1 is tert-butyl. The preferred R2 is phenyl, 3,5-dimethylphenyl, 2,4-dimethylphenyl, 3-methylphenyl, 4-methylphenyl, 2-methylphenyl, or 3,4-methylenedioxyphenyl. The preferred R3 is phenyl, 3-pyridyl, 3-methoxy-2-methylphenyl, 3-ethoxy-2-methylphenyl, 3-methoxy-2-ethylphenyl, 4-ethylphenyl, 2,6-difluorophenyl, 2,3-dimethylphenyl, 3-chloro-2-methylphenyl, or 3-bromo-2-methylphenyl. When R3, R4, and O are cyclized to form the cyclic ester known as a lactone, the lactone may be:

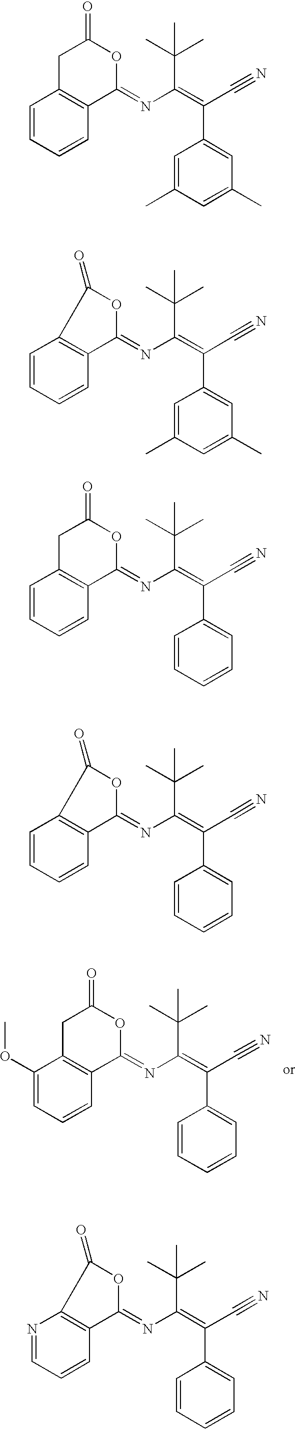

The compounds described as per Formula I and its tautomer, Formula II, and its isomer, Formula III, are useful for modulation of an exogenous gene in a living organism. They have the ability to activate or to suppress an exogenous gene.

Additionally, the described compounds can be used as pesticides, i.e., for arthropod control (control of segmented invertebrates such as insects, arachnids, crustaceans, or myriapods) on plants in soil or water, in structures, and on parasites in or on vertebrate animals, acting as agonists of 20-hydroxyecdysone, the molting hormone.

The preparation of these compounds may be accomplished by the following general scheme:

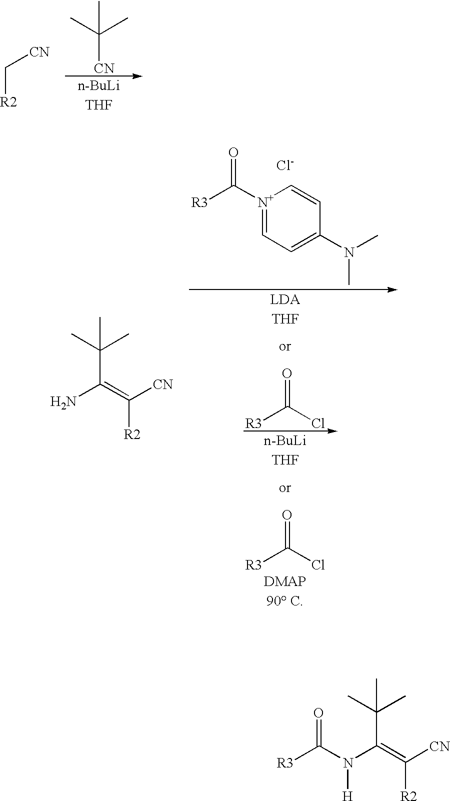

and in this scheme, R1 is tert-butyl, but that is not a requirement.

### LABORATORY EXAMPLES

### Example 1

Preparation of starting material; (E)-3-amino-2-(3,5-dimethylphenyl)-4,4-dimethylpent-2-enenitrile

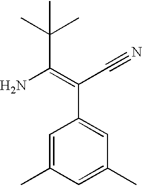

To 30 mL of n-butyllithium (2.5 M in hexane) at 5° C. was added 30 mL of dry tetrahydrofuran. The resulting solution was cooled back to 5° C. and a solution of 5 g of 3,5-dimethylphenylacetonitrile in 10 mL of tetrahydrofuran was added over 30 min., keeping the temperature between 5° C. and 10° C. The mixture was stirred at 5° C. for 1 h., and then a solution of 2.86 g of trimethylacetonitrile in 10 mL of tetrahydrofuran was added. The resulting mixture was stirred overnight at ambient temperature. The mixture was poured into ice water and extracted with 2 portions of ethyl acetate.

The combined ethyl acetate layers were washed with brine, dried over MgSO₄, filtered, and evaporated in vacuo to afford the crude product as an oil, which was crystallized from petroleum ether to yield 3.2 g of a solid with a ¹HNMR spectrum consistent with the expected product, namely (E)-3-amino-2-(3,5-dimethylphenyl)-4,4-dimethylpent-2-enenitrile.

Preparation of starting material; 3-methoxy-2-methylbenzoyl chloride

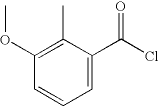

Thionyl chloride (5 mL) was gradually added to 0.95 g of 3-methoxy-2-methylbenzoic acid at room temperature. The resulting mixture was heated at 65° C. for 1 h. The excess thionyl chloride was evaporated in vacuo, and a small portion of carbon tetrachloride was added. Then, the mixture was again evaporated in vacuo to yield the desired product as an oil. This was used directly in the next reaction.

Preparation of N-[(E)-1-tert-butyl-2-cyano-2-(3,5-dimethylphenyl)-vinyl]-3-methoxy-2-methylbenzamide

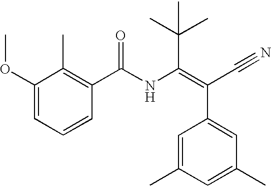

To 3.8 mL of lithium diisopropylamide solution (1.5 M in cyclohexane) at −78° C., was added dropwise, a solution of 0.51 g of (E)-3-amino-2-(3,5-dimethylphenyl)-4,4-dimethylpent-2-enenitrile in 30 mL of dry tetrahydrofuran. The mixture was stirred for 30 min. at −78° C., and then 1.05 g of 3-methoxy-2-methylbenzoyl chloride was added in one portion. The resulting mixture was stirred overnight at ambient temperature. The reaction mixture was poured into ice water and extracted with ethyl acetate.

The ethyl acetate layer was washed with brine, dried over MgSO₄, filtered, and evaporated in vacuo to yield 1.4 g of crude solid product. The crude product was partially purified using 3 plates, each being a 600 mm×20 mm silica gel preparative layer chromatography plate, eluted with 20% ethyl acetate in hexane to afford 0.17 g of slightly impure material. This was recrystallized from a 3 mL tetrahydrofuran and 15 mL hexane mixture to yield 0.15 g of white crystalline material (mp was 203 to 204° C.) with GC/MS and ¹HNMR spectra consistent with the desired product, namely N-[(E)-1-tert-butyl-2-cyano-2-(3,5-dimethylphenyl)-vinyl]-3-methoxy-2-methylbenzamide.

### Example 2

Using essentially the same procedure as described above, the following selected cyanoenamine compounds have also been prepared, as reported in Table A1 below.

TABLE A1

| | Melting point LC/MS |
|---|---|
| Compound | degrees C. molecular ion |
| 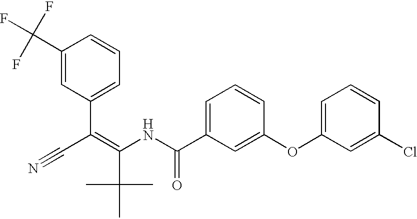 | 499 |
| Compound 1 | |
| 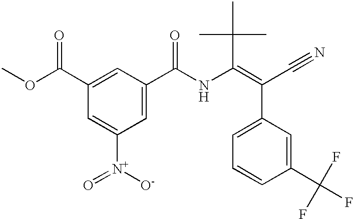 | 476 |
| Compound 2 | |
| 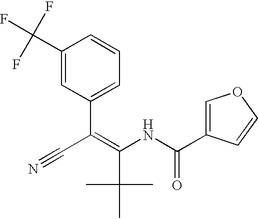 | 363 |
| Compound 3 | |
| 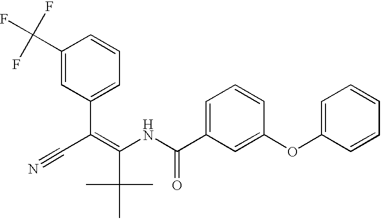 | 465 |
| Compound 4 | |
| 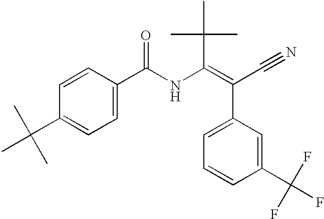 | 429 |
| Compound 5 | |
| 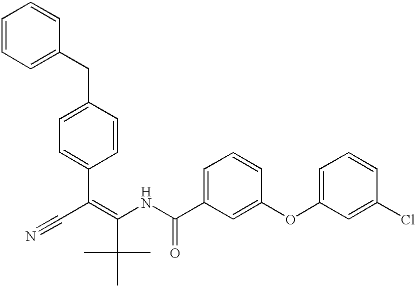 | 521 |
| Compound 6 | |
| 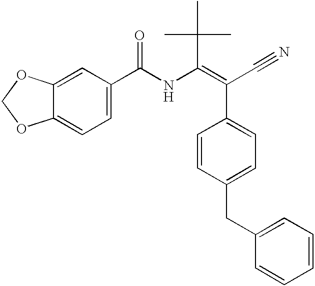 | 439 |
| Compound 7 | |
| 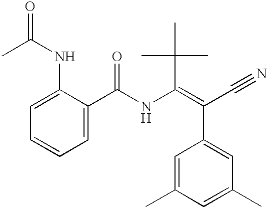 | 390 |
| Compound 8 | |
| 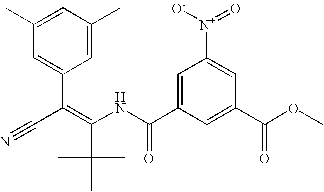 | 436 |
| Compound 9 | |
| 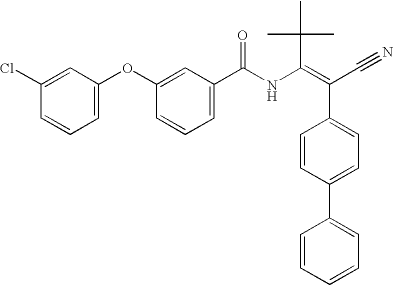 | 507 |
| Compound 10 | |
| 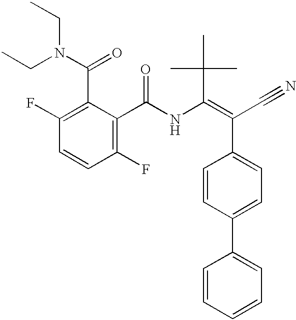 | 516 |
| Compound 11 | |
| 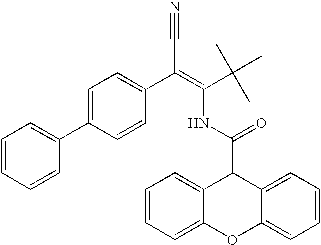 | 485 |
| Compound 12 | |
| 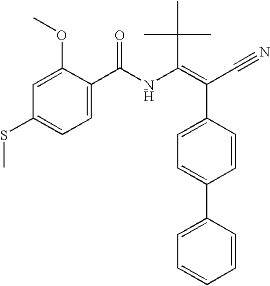 | 457 |
| Compound 13 | |
| 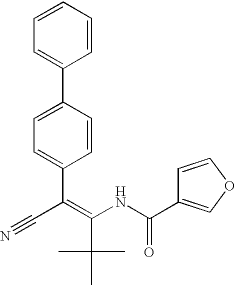 | 371 |
| Compound 14 | |
| 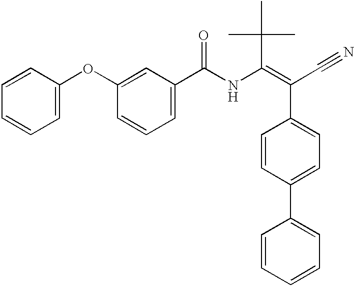 | 473 |
| Compound 15 | |
| 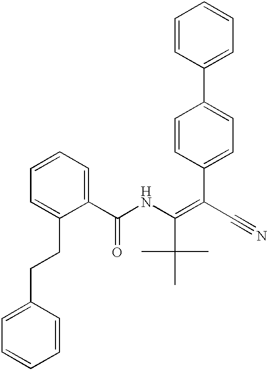 | 485 |
| Compound 16 | |
| 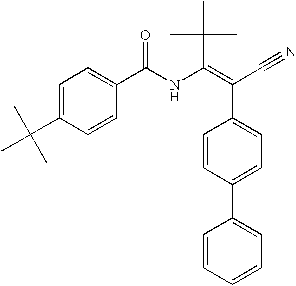 | 437 |
| Compound 17 | |
| 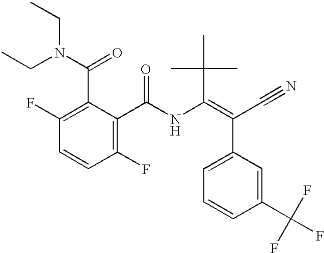 | 508 |
| Compound 18 | |
| 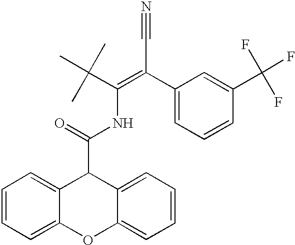 | 477 |
| Compound 19 | |
| 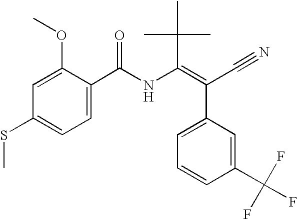 | 449 |
| Compound 20 | |
| 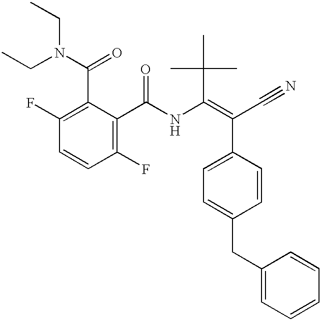 | 530 |
| Compound 21 | |
| 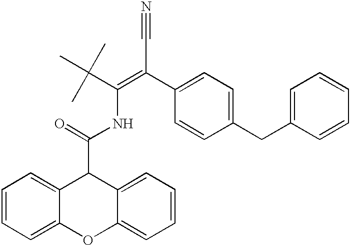 | 499 |
| Compound 22 | |
| 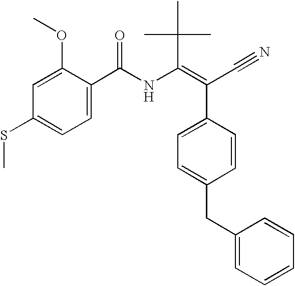 | 471 |
| Compound 23 | |
| 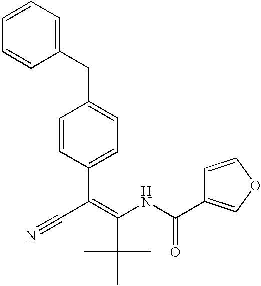 | 385 |
| Compound 24 | |
| 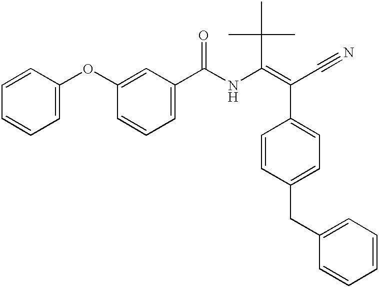 | 487 |
| Compound 25 | |
| 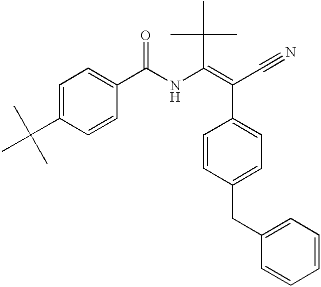 | 451 |
| Compound 26 | |
| 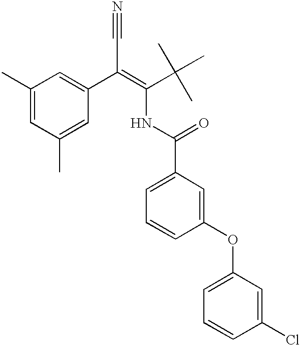 | 459 |
| Compound 27 | |
| 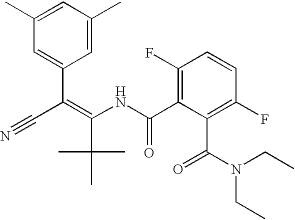 | 468 |
| Compound 28 | |
| 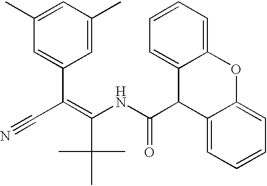 | 437 |
| Compound 29 | |
| 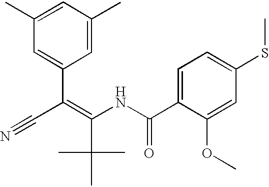 | 409 |
| Compound 30 | |
| 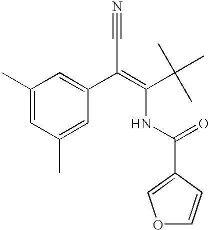 | 323 |
| Compound 31 | |
| 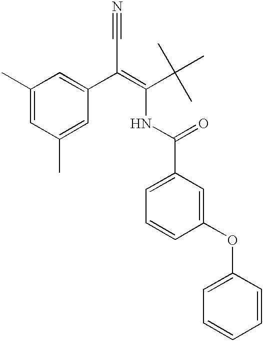 | 425 |
| Compound 32 | |
| 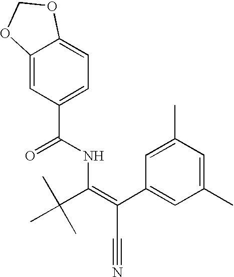 | 377 |
| Compound 33 | |
| 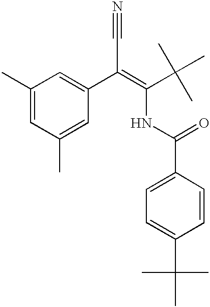 | 389 |
| Compound 34 | |
| 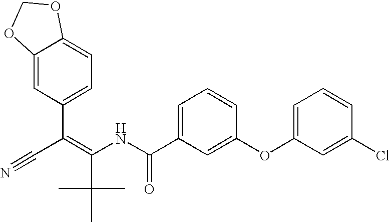 | 475 |
| Compound 35 | |
| 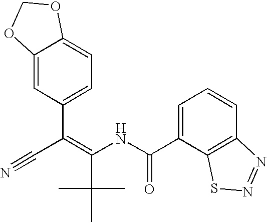 | 407 |
| Compound 36 | |
| 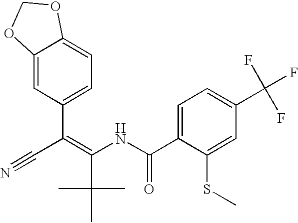 | 463 |
| Compound 37 | |
| 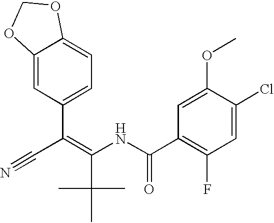 | 431 |
| Compound 38 | |
| 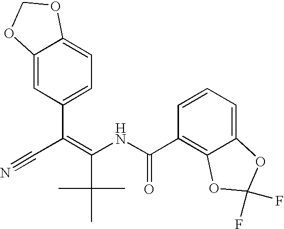 | 429 |
| Compound 39 | |
| 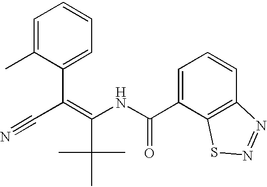 | 377 |
| Compound 40 | |
| 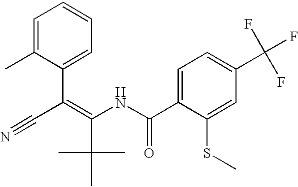 | 433 |
| Compound 41 | |
| 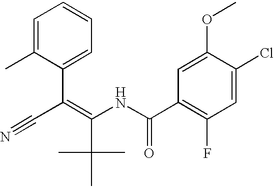 | 401 |
| Compound 42 | |
| 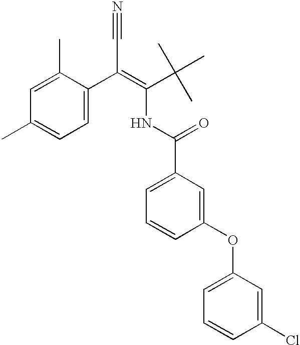 | 459 |
| Compound 43 | |
| 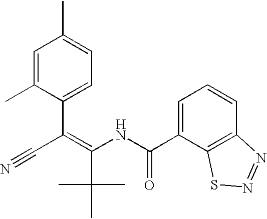 | 391 |
| Compound 44 | |
| 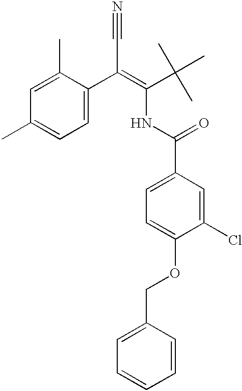 | 473 |
| Compound 45 | |
| 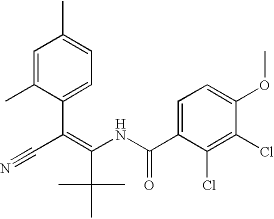 | 431 |
| Compound 46 | |
| 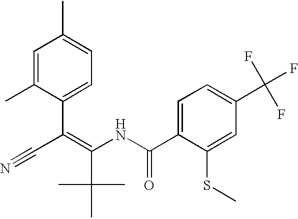 | 447 |
| Compound 47 | |
| 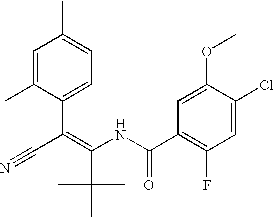 | 415 |
| Compound 48 | |
| 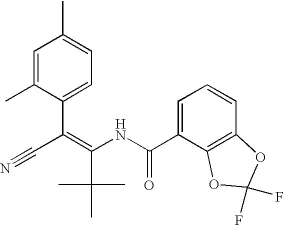 | 413 |
| Compound 49 | |
| 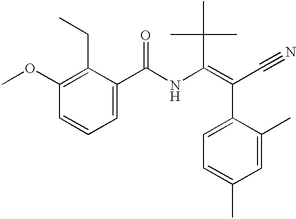 | 391 |
| Compound 50 | |
| 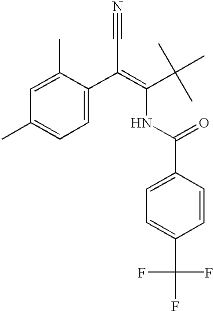 | 401 |
| Compound 51 | |
| 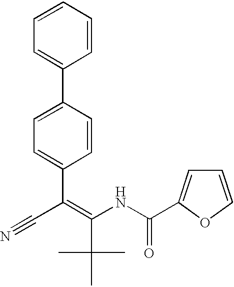 | 371 |
| Compound 52 | |
| 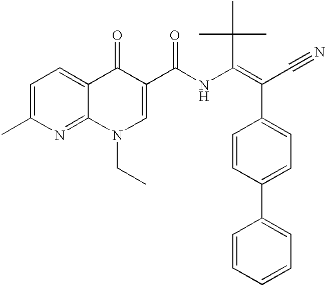 | 491 |
| Compound 53 | |
| 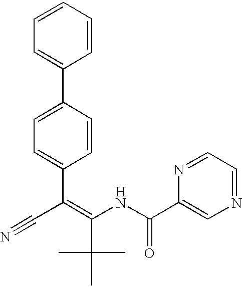 | 383 |
| Compound 54 | |
| 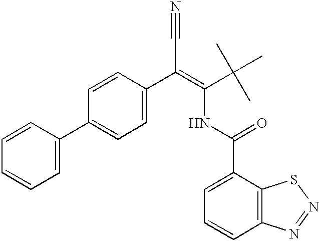 | 439 |
| Compound 55 | |
| 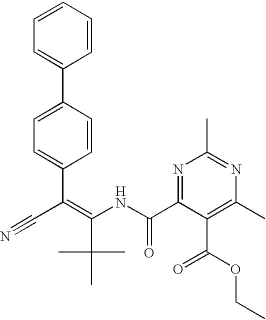 | 483 |
| Compound 56 | |
| 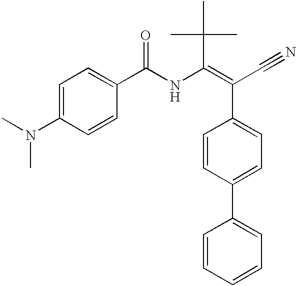 | 424 |
| Compound 57 | |
| 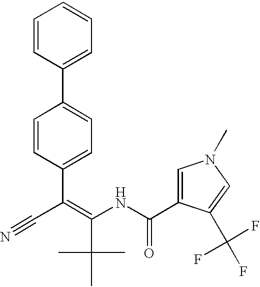 | 452 |
| Compound 58 | |
| 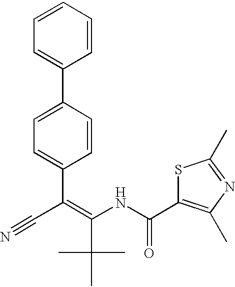 | 416 |
| Compound 59 | |
| 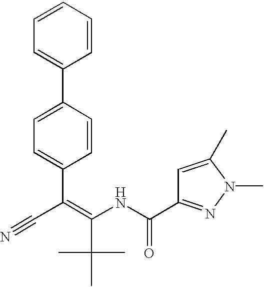 | 399 |
| Compound 60 | |
| 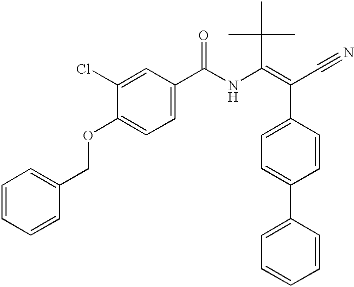 | 521 |
| Compound 61 | |
| 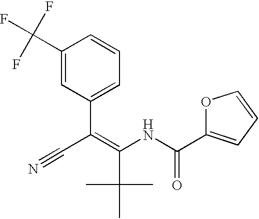 | 363 |
| Compound 62 | |
| 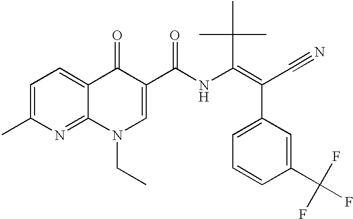 | 483 |
| Compound 63 | |
| 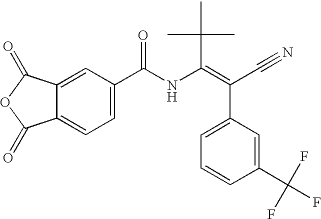 | 443 |
| Compound 64 | |
| 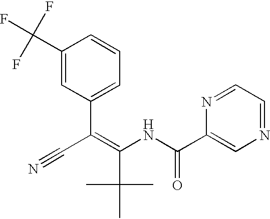 | 375 |
| Compound 65 | |
| 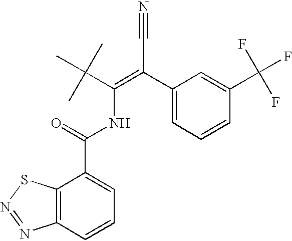 | 431 |
| Compound 66 | |
| 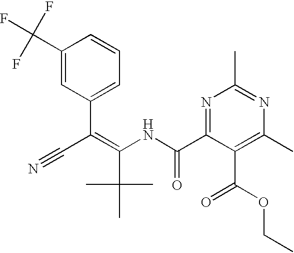 | 475 |
| Compound 67 | |
| 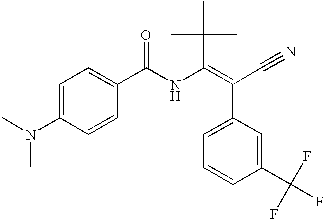 | 416 |
| Compound 68 | |
| 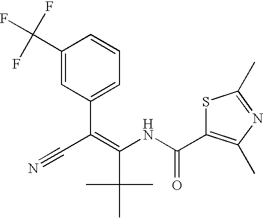 | 408 |
| Compound 69 | |
| 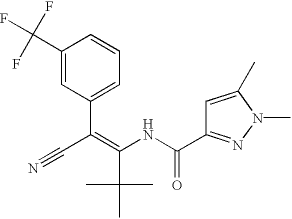 | 391 |
| Compound 70 | |
| 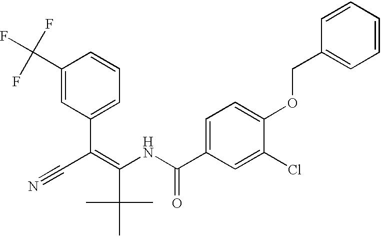 | 513 |
| Compound 71 | |
| 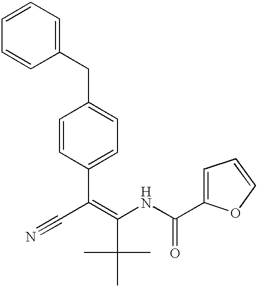 | 385 |
| Compound 72 | |
| 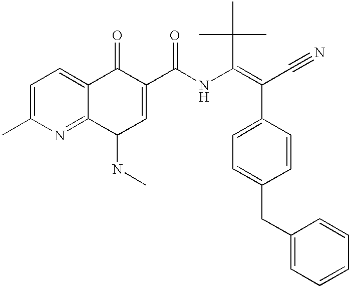 | 505 |
| Compound 73 | |
| 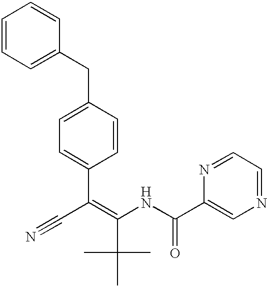 | 397 |
| Compound 74 | |
| 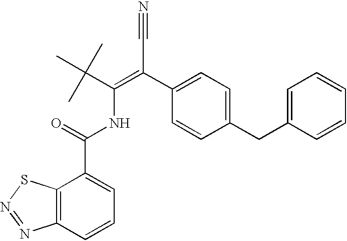 | 453 |
| Compound 75 | |
| 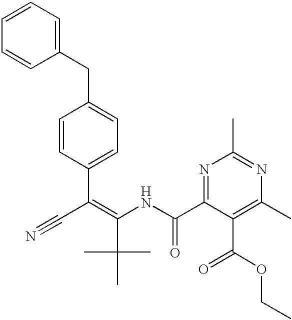 | 497 |
| Compound 76 | |
| 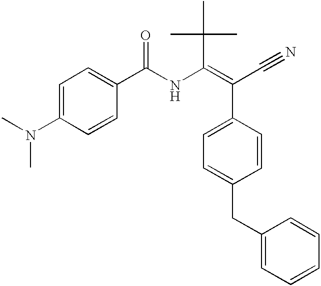 | 438 |
| Compound 77 | |
| 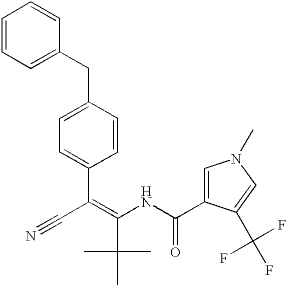 | 466 |
| Compound 78 | |
| 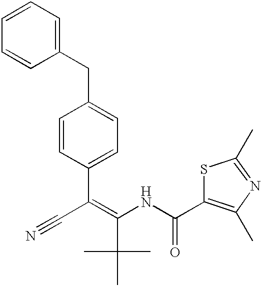 | 430 |
| Compound 79 | |
| 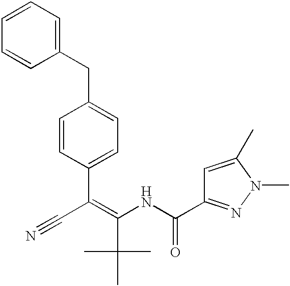 | 413 |
| Compound 80 | |
| 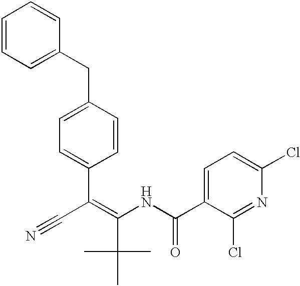 | 464 |
| Compound 81 | |
| 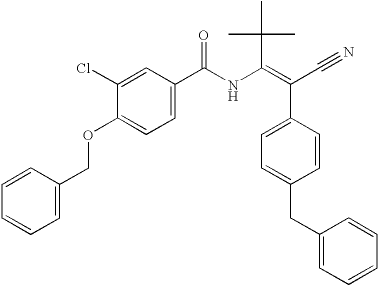 | 535 |
| Compound 82 | |
| 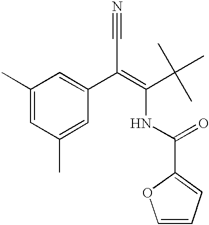 | 323 |
| Compound 83 | |
| 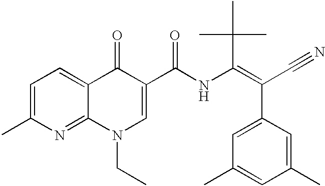 | 443 |
| Compound 84 | |
| 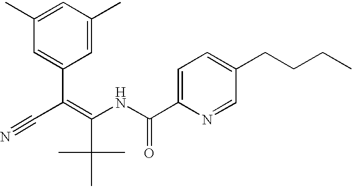 | 390 |
| Compound 85 | |
| 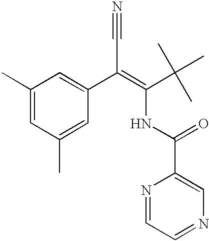 | 335 |
| Compound 86 | |
| 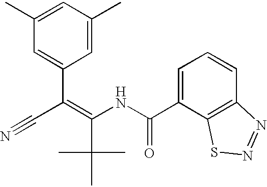 | 391 |
| Compound 87 | |
| 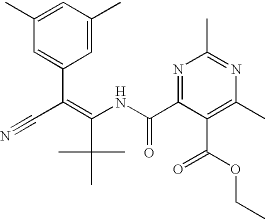 | 435 |
| Compound 88 | |
| 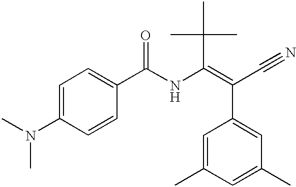 | 376 |
| Compound 89 | |
| 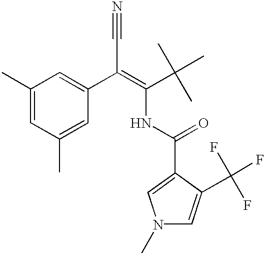 | 404 |
| Compound 90 | |
| 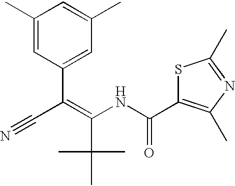 | 368 |
| Compound 91 | |
| 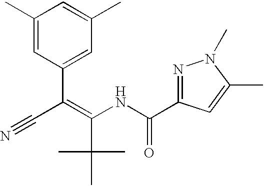 | 351 |
| Compound 92 | |
| 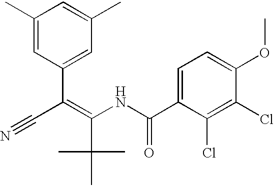 | 431 |
| Compound 93 | |
| 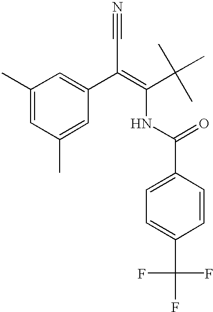 | 401 |
| Compound 94 | |
| 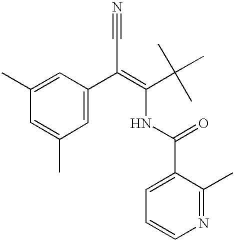 | 120 |
| Compound 95 | |
| 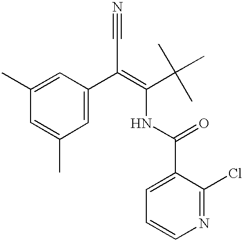 | 165 |
| Compound 96 | |
| 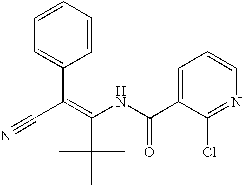 | 170 |
| Compound 97 | |
| 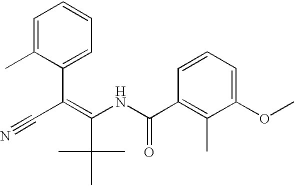 | 128-133 |
| Compound 98 | |
| 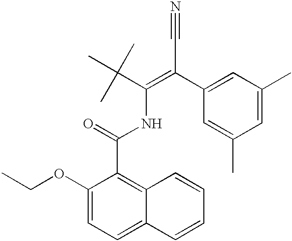 | 165 |
| Compound 99 | |
| 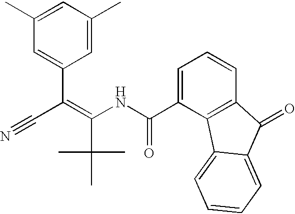 | 195 |
| Compound 100 | |
| 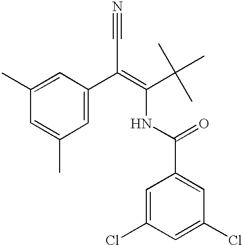 | 145 |
| Compound 101 | |
| 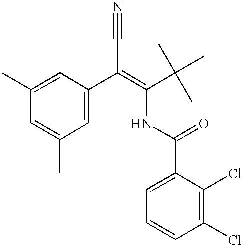 | 190 |
| Compound 102 | |
| 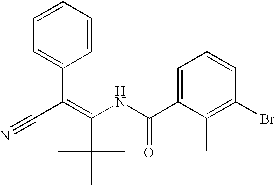 | 150 |
| Compound 103 | |
| 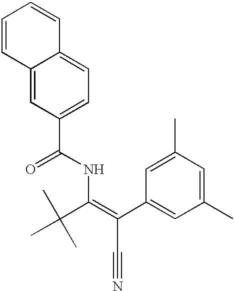 | 198 |
| Compound 104 | |
| 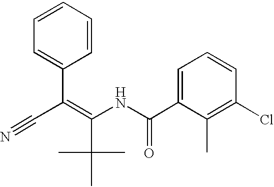 | 153 |
| Compound 105 | |
| 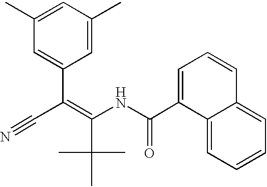 | 205 |
| Compound 106 | |
| 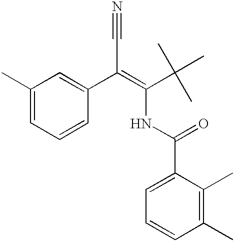 | 130 |
| Compound 107 | |
| 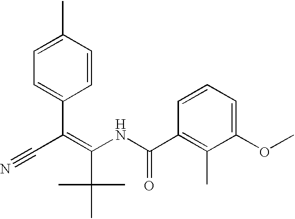 | 165 |
| Compound 108 | |
| 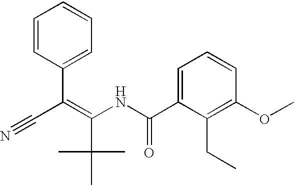 | 165 |
| Compound 109 | |
| 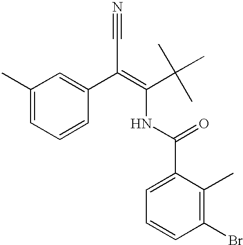 | 82.1 |
| Compound 110 | |
| 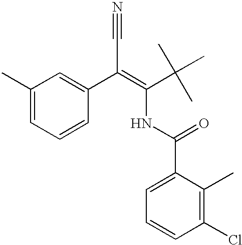 | 125 |
| Compound 111 | |
| 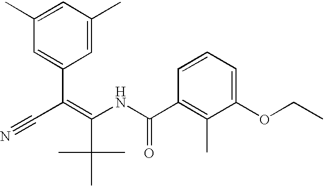 | 170 |
| Compound 112 | |
| 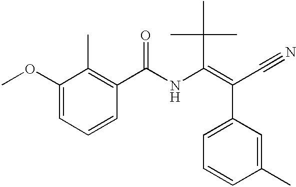 | 145 |
| Compound 113 | |
| 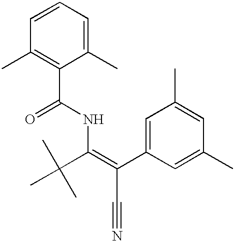 | 160 |
| Compound 114 | |
| 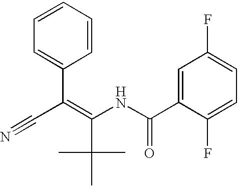 | 125 |
| Compound 115 | |
| 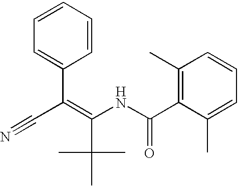 | 220 |
| Compound 116 | |
| 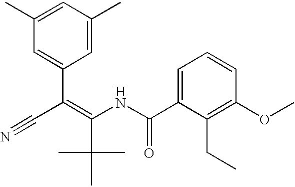 | 391 |
| Compound 117 | |
| 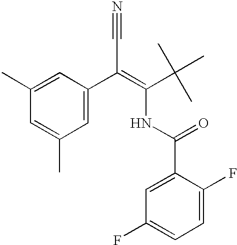 | 179-182 |
| Compound 118 | |
| 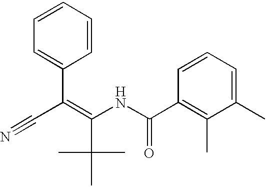 | 175 |
| Compound 119 | |
| 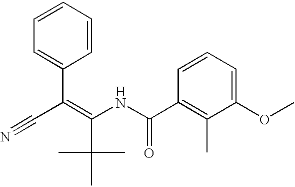 | 170 |
| Compound 120 | |
| 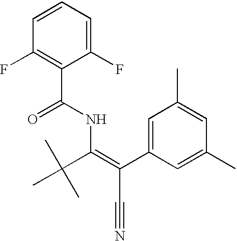 | 225 |
| Compound 121 | |
| 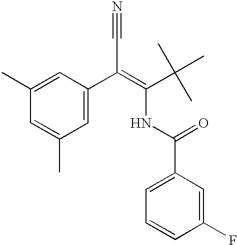 | 184.4 |
| Compound 122 | |
| 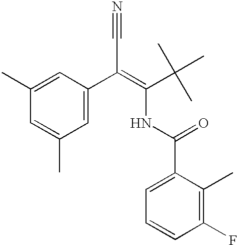 | 125.9 |
| Compound 123 | |
| 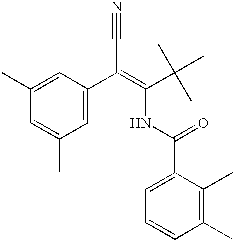 | 171-174 |
| Compound 124 | |
| 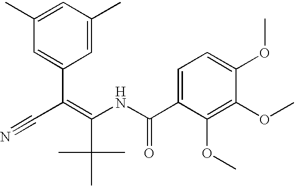 | 113.6 |
| Compound 125 | |
| 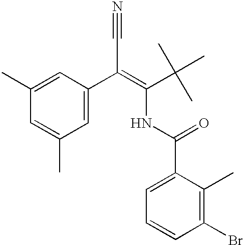 | >300 |
| Compound 126 | |
| 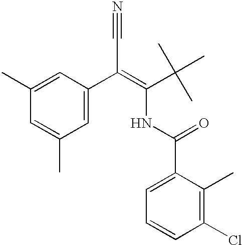 | 194.7 |
| Compound 127 | |
| 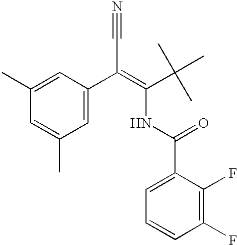 | 190 |
| Compound 128 | |
| 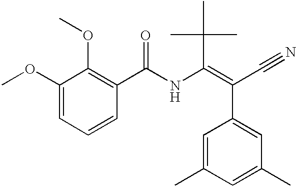 | 393 |
| Compound 129 | |
| 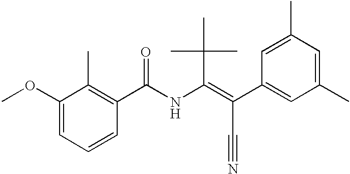 | >300 |
| Compound 130 | |
| 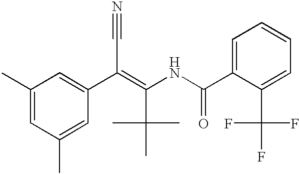 | 178-180 |
| Compound 131 | |
| 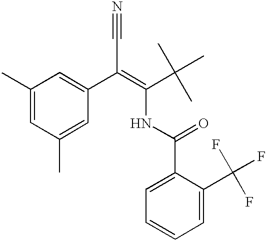 | 197-200 |
| Compound 132 | |
| 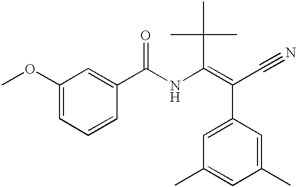 | 203-205 |
| Compound 133 | |
| 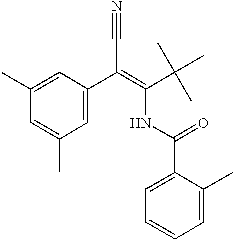 | 178-180 |
| Compound 134 | |
| 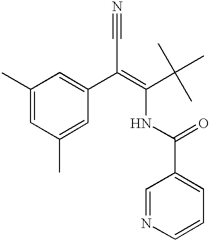 | 173-174 |
| Compound 135 | |
| 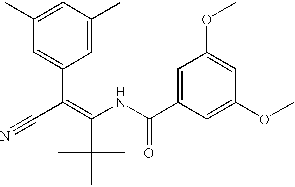 | 194-195 |
| Compound 136 | |
| 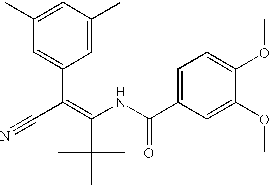 | 194-195 |
| Compound 137 | |
| 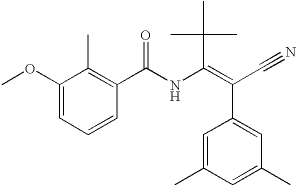 | 203-204 |
| Compound 138 | |
| 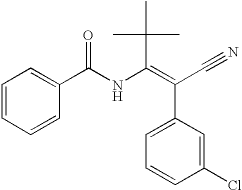 | 146-147 |
| Compound 139 | |
| 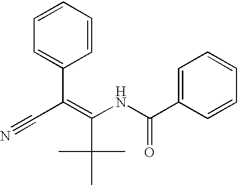 | 142-144 |
| Compound 140 | |
| 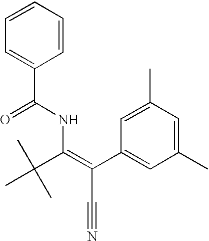 | 175-176 |
| Compound 141 | |
| 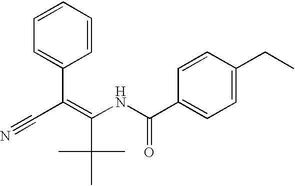 | 138-140 |
| Compound 142 | |
| 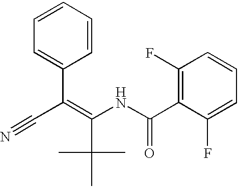 | 180-183 |
| Compound 143 | |
| 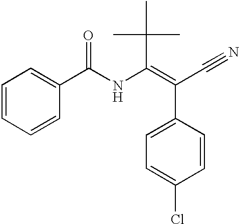 | 219-222 |
| Compound 144 | |

The various R1, R2, R3, and R4 moieties (from the compounds made as per Table A1 above) are summarized in Table A2 below.

TABLE A2

| Compound | | | | |
|---|---|---|---|---|
| # | R1 | R2 | R3 | R4 |
| 1 | t-Bu | 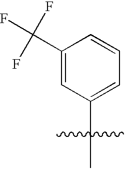 | 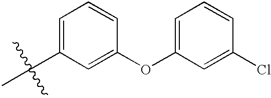 | H |
| 2 | t-Bu | 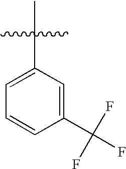 | 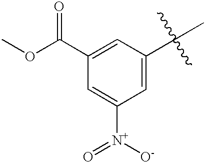 | H |
| 3 | t-Bu | 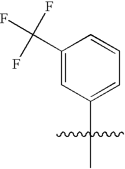 | 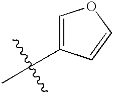 | H |
| 4 | t-Bu | 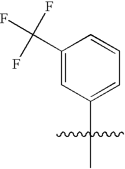 | 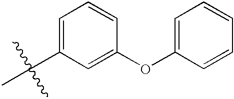 | H |
| 5 | t-Bu | 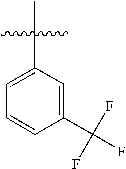 | 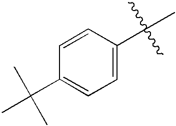 | H |
| 6 | t-Bu | 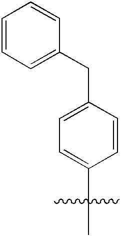 | 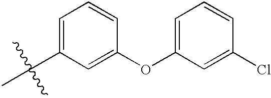 | H |
| 7 | t-Bu | 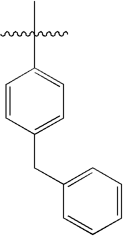 | 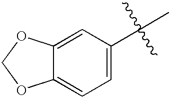 | H |
| 8 | t-Bu | 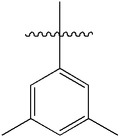 | 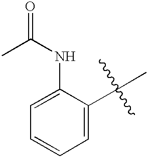 | H |
| 9 | t-Bu | 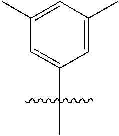 | 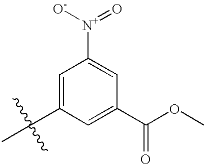 | H |
| 10 | t-Bu | 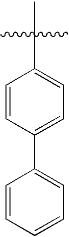 | 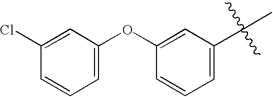 | H |
| 11 | t-Bu | 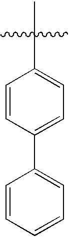 | 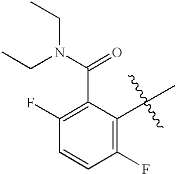 | H |
| 12 | t-Bu | 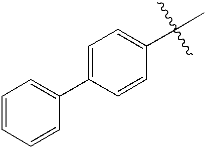 | 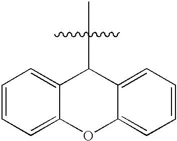 | H |
| 13 | t-Bu | 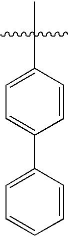 | 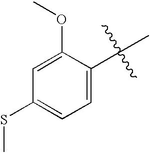 | H |
| 14 | t-Bu | 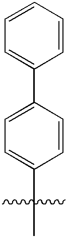 | 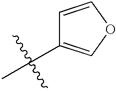 | H |
| 15 | t-Bu | 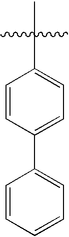 | 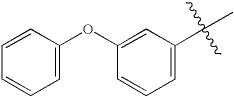 | H |
| 16 | t-Bu | 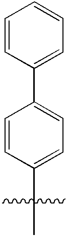 | 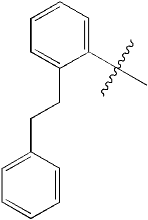 | H |
| 17 | t-Bu | 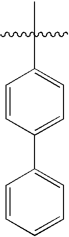 | 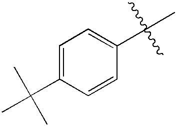 | H |
| 18 | t-Bu | 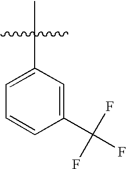 | 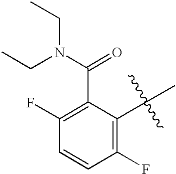 | H |
| 19 | t-Bu | 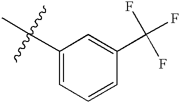 | 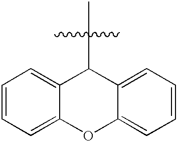 | H |
| 20 | t-Bu | 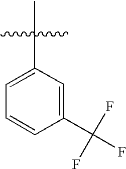 | 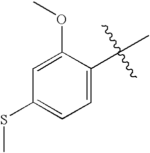 | H |
| 21 | t-Bu | 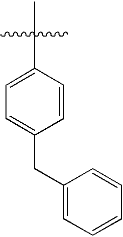 | 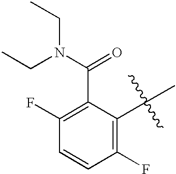 | H |
| 22 | t-Bu | 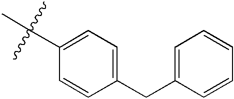 | 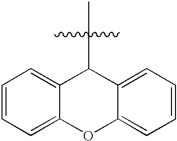 | H |
| 23 | t-Bu | 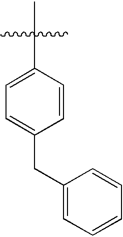 | 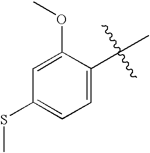 | H |
| 24 | t-Bu | 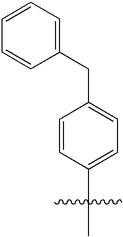 | 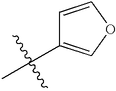 | H |
| 25 | t-Bu | 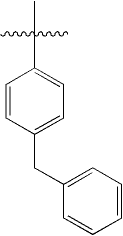 | 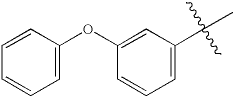 | H |
| 26 | t-Bu | 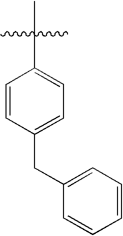 | 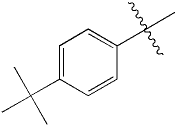 | H |
| 27 | t-Bu | 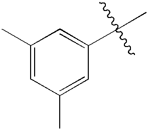 | 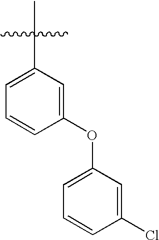 | H |
| 28 | t-Bu | 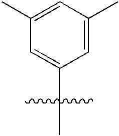 | 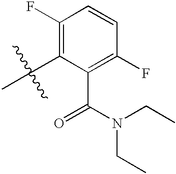 | H |
| 29 | t-Bu | 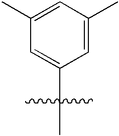 | 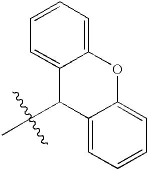 | H |
| 30 | t-Bu | 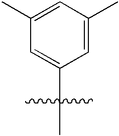 | 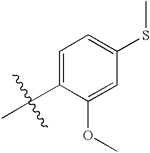 | H |
| 31 | t-Bu | 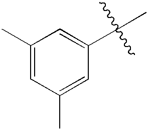 | 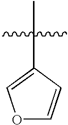 | H |
| 32 | t-Bu | 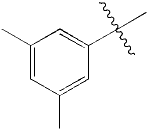 | 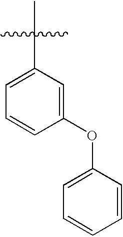 | H |
| 33 | t-Bu | 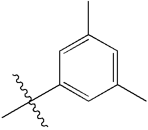 | 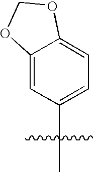 | H |
| 34 | t-Bu | 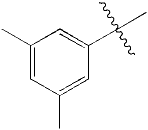 | 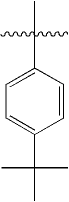 | H |
| 35 | t-Bu | 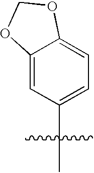 | 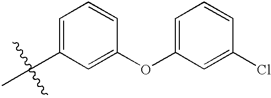 | H |
| 36 | t-Bu | 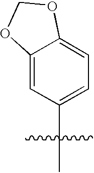 | 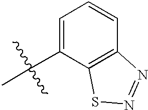 | H |
| 37 | t-Bu | 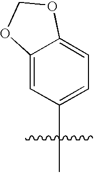 | 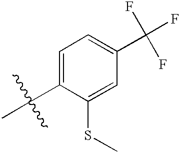 | H |
| 38 | t-Bu | 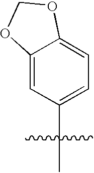 | 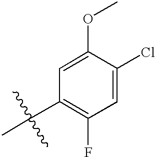 | H |
| 39 | t-Bu | 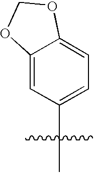 | 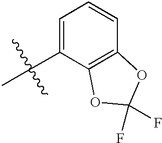 | H |
| 40 | t-Bu | 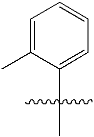 | 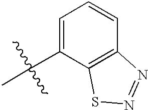 | H |
| 41 | t-Bu | 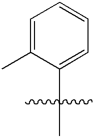 | 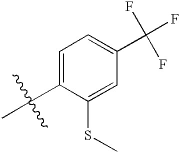 | H |
| 42 | t-Bu | 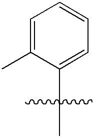 | 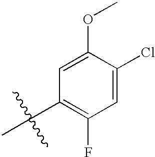 | H |
| 43 | t-Bu | 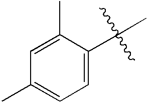 | 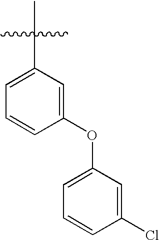 | H |
| 44 | t-Bu | 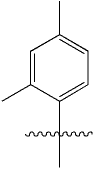 | 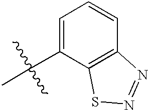 | H |
| 45 | t-Bu | 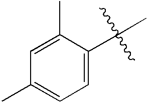 | 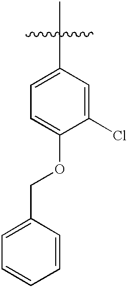 | H |
| 46 | t-Bu | 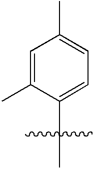 | 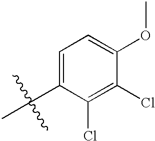 | H |
| 47 | t-Bu | 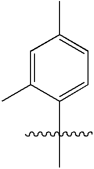 | 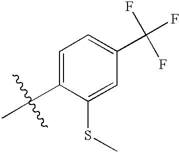 | H |
| 48 | t-Bu | 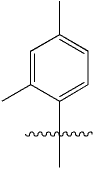 | 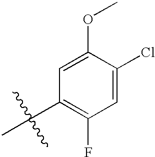 | H |
| 49 | t-Bu | 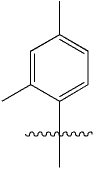 | 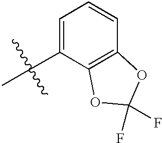 | H |
| 50 | t-Bu | 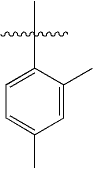 | 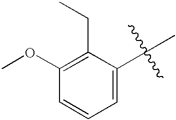 | H |
| 51 | t-Bu | 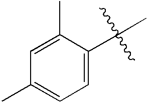 | 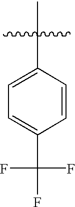 | H |
| 52 | t-Bu | 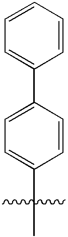 | 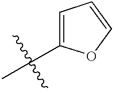 | H |
| 53 | t-Bu | 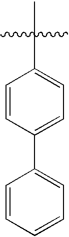 | 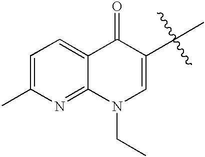 | H |
| 54 | t-Bu | 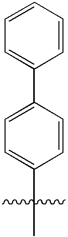 | 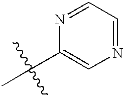 | H |
| 55 | t-Bu | 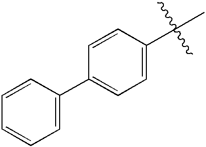 | 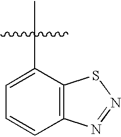 | H |
| 56 | t-Bu | 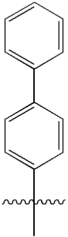 | 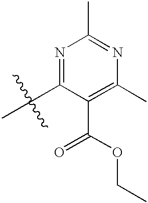 | H |
| 57 | t-Bu | 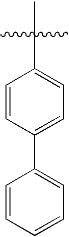 | 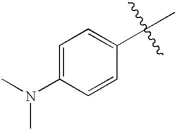 | H |
| 58 | t-Bu | 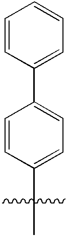 | 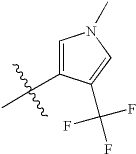 | H |
| 59 | t-Bu | 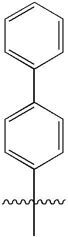 | 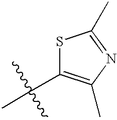 | H |
| 60 | t-Bu | 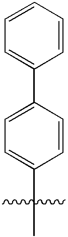 | 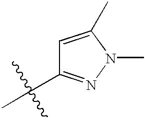 | H |
| 61 | t-Bu | 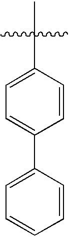 | 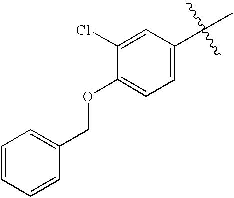 | H |
| 62 | t-Bu | 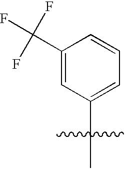 | 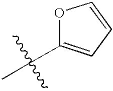 | H |
| 63 | t-Bu | 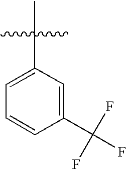 | 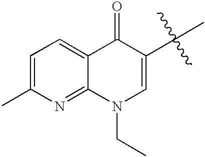 | H |
| 64 | t-Bu | 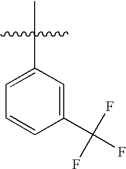 | 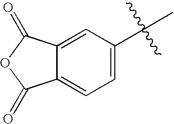 | H |
| 65 | t-Bu | 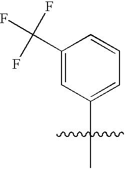 | 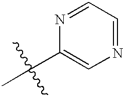 | H |
| 66 | t-Bu | 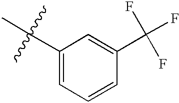 | 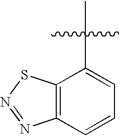 | H |
| 67 | t-Bu | 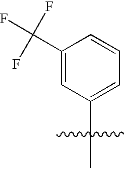 | 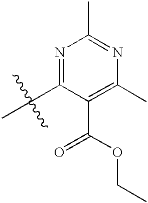 | H |
| 68 | t-Bu | 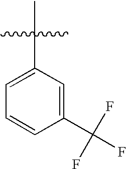 | 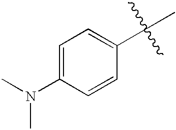 | H |
| 69 | t-Bu | 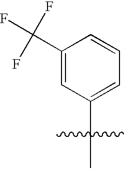 | 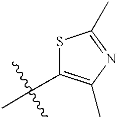 | H |
| 70 | t-Bu | 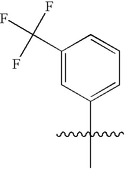 | 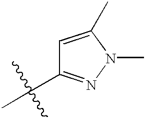 | H |
| 71 | t-Bu | 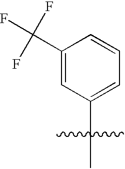 | 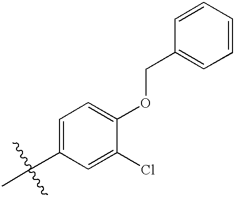 | H |
| 72 | t-Bu | 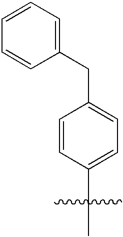 | 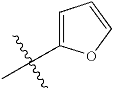 | H |
| 73 | t-Bu | 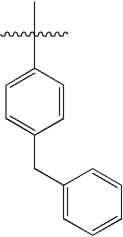 | 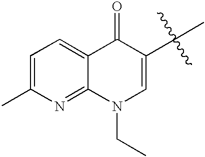 | H |
| 74 | t-Bu | 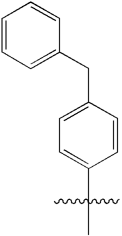 | 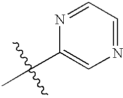 | H |
| 75 | t-Bu | 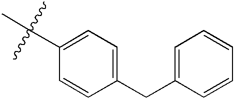 | 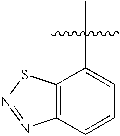 | H |
| 76 | t-Bu | 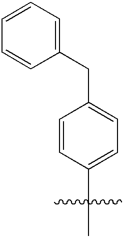 | 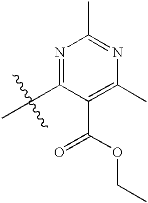 | H |
| 77 | t-Bu | 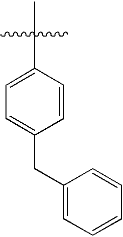 | 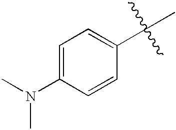 | H |
| 78 | t-Bu | 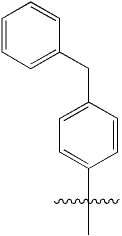 | 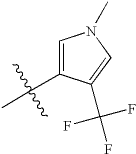 | H |
| 79 | t-Bu | 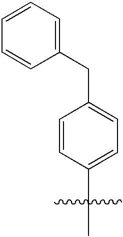 | 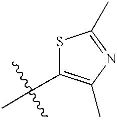 | H |
| 80 | t-Bu | 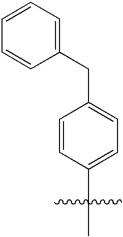 | 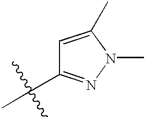 | H |
| 81 | t-Bu | 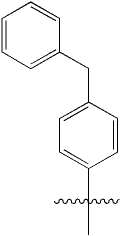 | 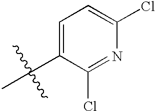 | H |
| 82 | t-Bu | 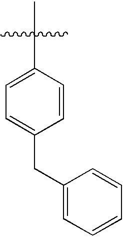 | 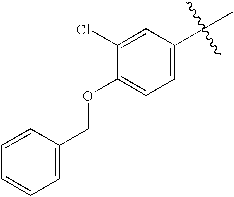 | H |
| 83 | t-Bu | 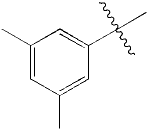 | 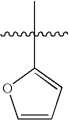 | H |
| 84 | t-Bu | 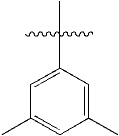 | 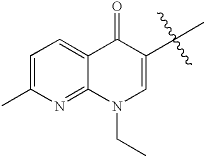 | H |
| 85 | t-Bu | 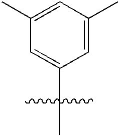 | 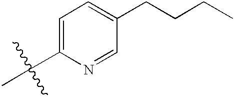 | H |
| 86 | t-Bu | 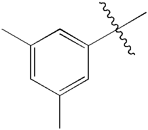 | 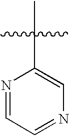 | H |
| 87 | t-Bu | 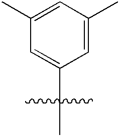 | 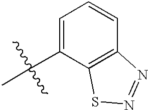 | H |
| 88 | t-Bu | 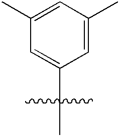 | 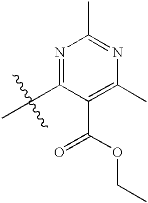 | H |
| 89 | t-Bu | 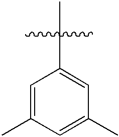 | 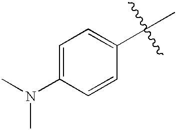 | H |
| 90 | t-Bu | 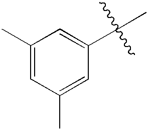 | 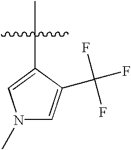 | H |
| 91 | t-Bu | 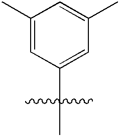 | 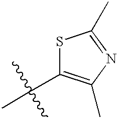 | H |
| 92 | t-Bu | 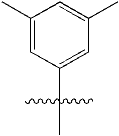 | 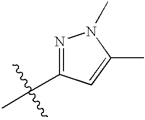 | H |
| 93 | t-Bu | 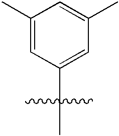 | 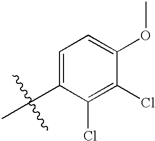 | H |
| 94 | t-Bu | 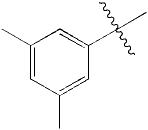 | 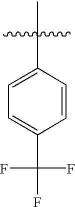 | H |
| 95 | t-Bu | 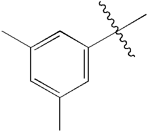 | 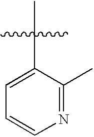 | H |
| 96 | t-Bu | 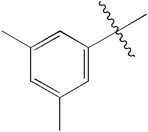 | 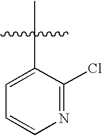 | H |
| 97 | t-Bu | 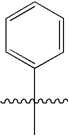 | 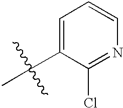 | H |
| 98 | t-Bu | 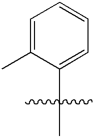 | 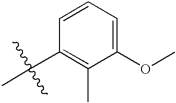 | H |
| 99 | t-Bu | 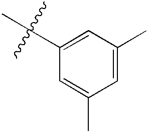 | 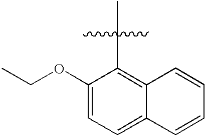 | H |
| 100 | t-Bu | 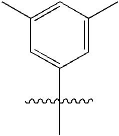 | 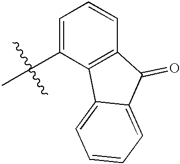 | H |
| 101 | t-Bu | 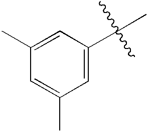 | 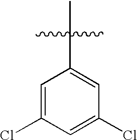 | H |
| 102 | t-Bu | 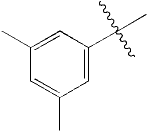 | 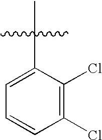 | H |
| 103 | t-Bu | 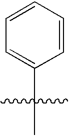 | 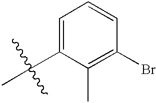 | H |
| 104 | t-Bu | 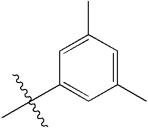 | 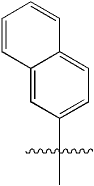 | H |
| 105 | t-Bu | 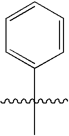 | 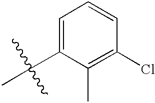 | H |
| 106 | t-Bu | 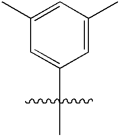 | 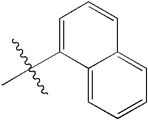 | H |
| 107 | t-Bu | 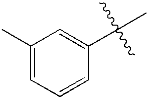 | 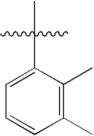 | H |
| 108 | t-Bu | 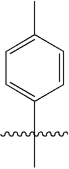 | 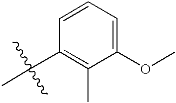 | H |
| 109 | t-Bu | 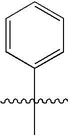 | 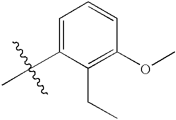 | H |
| 110 | t-Bu | 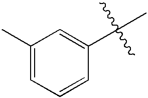 | 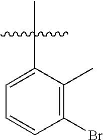 | H |
| 111 | t-Bu | 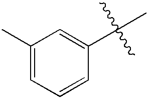 | 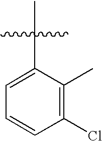 | H |
| 112 | t-Bu | 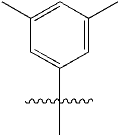 | 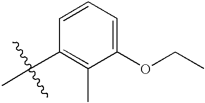 | H |
| 113 | t-Bu | 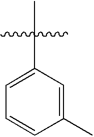 | 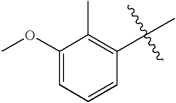 | H |
| 114 | t-Bu | 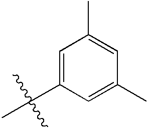 | 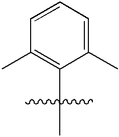 | H |
| 115 | t-Bu | 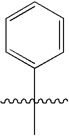 | 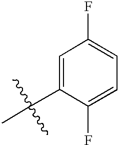 | H |
| 116 | t-Bu | 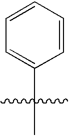 | 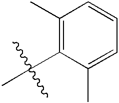 | H |
| 117 | t-Bu | 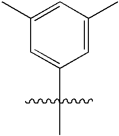 | 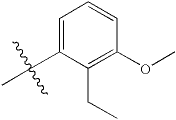 | H |
| 118 | t-Bu | 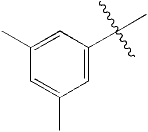 | 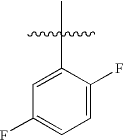 | H |
| 119 | t-Bu | 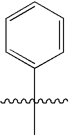 | 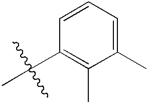 | H |
| 120 | t-Bu | 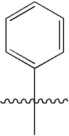 | 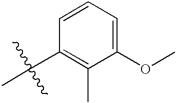 | H |
| 121 | t-Bu | 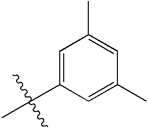 | 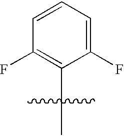 | H |
| 122 | t-Bu | 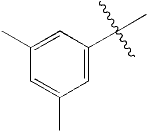 | 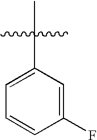 | H |
| 123 | t-Bu | 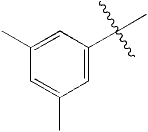 | 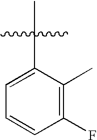 | H |
| 124 | t-Bu | 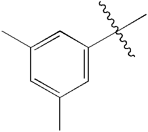 | 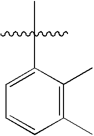 | H |
| 125 | t-Bu | 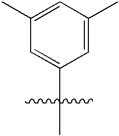 | 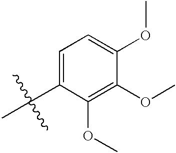 | H |
| 126 | t-Bu | 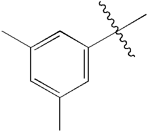 | 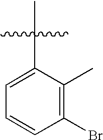 | H |
| 127 | t-Bu | 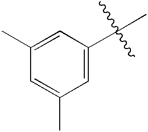 | 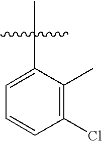 | H |
| 128 | t-Bu | 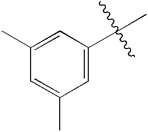 | 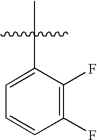 | H |
| 129 | t-Bu | 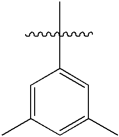 | 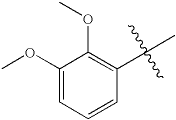 | H |
| 130 | t-Bu | 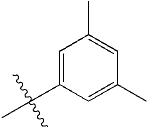 | 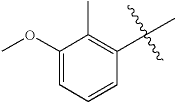 | H |
| 131 | t-Bu | 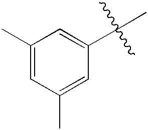 | 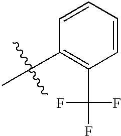 | H |
| 132 | t-Bu | 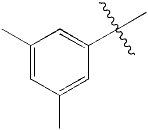 | 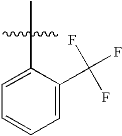 | H |
| 133 | t-Bu | 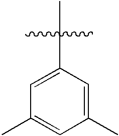 | 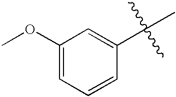 | H |
| 134 | t-Bu | 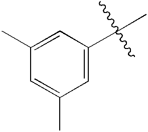 | 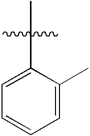 | H |
| 135 | t-Bu | 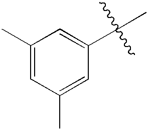 | 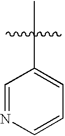 | H |
| 136 | t-Bu | 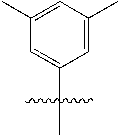 | 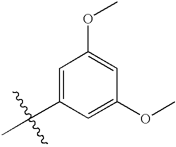 | H |
| 137 | t-Bu | 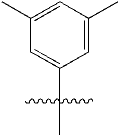 | 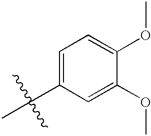 | H |
| 138 | t-Bu | 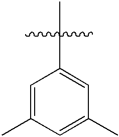 | 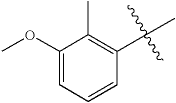 | H |
| 139 | t-Bu | 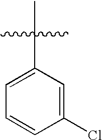 | 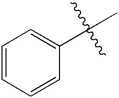 | H |
| 140 | t-Bu | 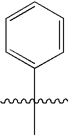 | 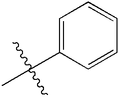 | H |
| 141 | t-Bu | 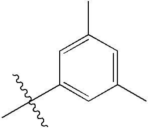 | 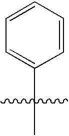 | H |
| 142 | t-Bu | 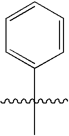 | 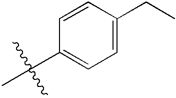 | H |
| 143 | t-Bu | 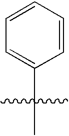 | 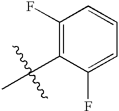 | H |
| 144 | t-Bu | 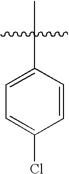 | 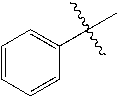 | H |

### Example 3

The following cyanoenamine compounds have been tested for pesticidal activity, according to the following procedures.

Spodoptera littoralis (abbreviated as SPODLI) (commonly known as Egyptian cotton leafworm): larvicide, feeding/contact activity. Cotton leaf discs were placed on agar in petri dishes and individually sprayed with test solution of cyanoenamine in an application chamber. After drying, the leaf discs were infested with 20 to 25 L1 larvae. The samples were checked for mortality, repellent effect, feeding behavior, and growth regulation 2 and 6 days after treatment.

Heliothis virescens (abbreviated as Hv) (commonly known as tobacco budworm): ovo-larvicide, feeding/contact activity. 30 to 35 fresh eggs (0 to 24 hours old), deposited on filter paper, were placed in petri dishes on a layer of artificial diet and 0.8 mL of each test solution of cyanoenamine was individually pipetted onto them. After an incubation period of 6 days, samples were checked for egg mortality, larval mortality, and growth regulation.

Each of Spodopertera littoralis and Heliothis virescens is a larval form of an insect in the order Lepidoptera.

The results are summarized in Table B below.

TABLE B

| | Insecticidal activity | |
|---|---|---|
| | (EC80 ppm) | |
| COMPOUND # | SPODLI | Hv |
| 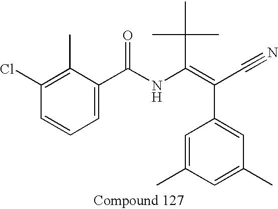 | 50 | 5 |
| 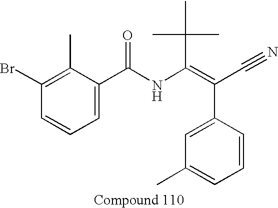 | 200 | 200 |
| 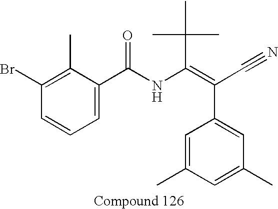 | >50 | >50 |
| 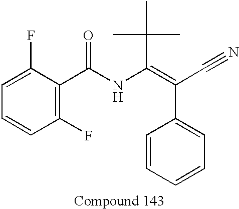 | >>100 | >100 |
| 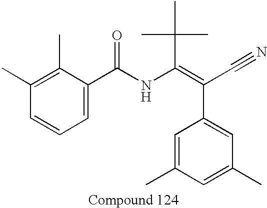 | 50 | 50 |
| 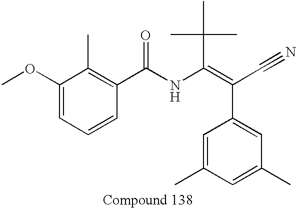 | 100 | 100 |
| 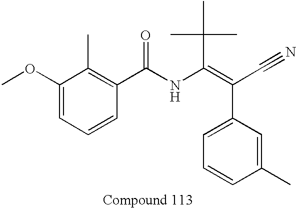 | 200 | 200 |
| 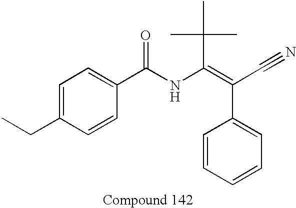 | >>100 | >>100 |

### Example 4

Construction of Reporter Plasmid

A minimal promoter vector was made by ligating a synthetic TATA box sequence oligonucleotide pair, 5′-agcttgagggtataatg-3′ (SEQ ID NO: 1) and 3′-actcccatattactcga-5′ (SEQ ID NO:2), into the HindIII site of vector pGL3-basic (Promega) so that the HindIII site was recreated 5′ to the inserted oligonucleotide and destroyed between the oligonucleotide and the downstream luciferase gene. This vector was designated TATA5.

The binding site from the hsp27 gene (Koelle et al., Cell 67(1): 59-77 (1991)) was made with the oligonucleotide pair, 5′-gatccgagacaagggttcaatgcacttgtccaatga-3′ (SEQ ID NO:3) and 3′-gctctgttcccaagttacgtgaacaggttactctag-5′ (SEQ ID NO:4). This site was multimerized and ligated into the BglII site of vector TATA-5. One isolate, pCGS154, contained the sequence below in the inserted region, having 2 pairs of sites in inverted orientations. One site had a deletion of a single base from the consensus sequence. The sequence of the inserted region in pCGS154 is shown below:

| | |
|---|---|
| 1 | gatccgagac aagggttcaa tgcacttgtc caatgagatc(SEQ ID NO:5) |
| 41 | cgagacaagg gttcaatgca cttgtccaat gagatctcat |
| 81 | tggacaagtg cattgaacct tgtctcggat ctcattggac |
| 121 | aagtgcattg aacccttgtc tcggatc |

Cloning of EcR Receptor Plasmid

PCR primers were designed based on the published sequence for Manduca Sexta ecdysone receptor (EcR) (genbank accession number U19812 (SEQ ID Nos:6 and 7) to clone the gene in two halves. RNA was prepared from prepupae larva of Manduca sexta using the LiCl/phenol method (Current Protocols in Molecular Biology, Vol. 1, Unit 4.3, 1987, John Wiley and Sons, publishers) and 1 μg of total RNA was used to prepare cDNA using MMLV reverse transcriptase (Promega). The cDNA was used in a PCR reaction with the primers described above to generate two PCR products for the 5′ and 3′ halves of the gene. These were subcloned into the pGEM-TA vector (Promega) and sequenced. The two fragments were joined at a unique Ndel site within each fragment and ligated into pBS-KS (Stratagene) to create a full length Manduca sexta EcR clone named pBSFLMa. A HindIII site followed by an inframe stop codon and BamHI site was placed at the 3′ end of the E domain (ligand binding domain) of the Manduca EcR receptor using the oligonucleotide: 5′-ggatcctaaagcttcgtcgtcgacacttcg-3′ (SEQ ID NO:8).

A truncated Manduca EcR containing domains C, D and E of the receptor was constructed as follows. A BamHI site and in-frame ATG was engineered just 5′ to the C domain using the degenerate primers 5′-ggatccatgggycgagaagaattrtcaccr-3′ (SEQ ID NO:9) and 5′-ccacrtcccagatctcctcga-3′ (SEQ ID NO:10). This fragment was then joined using the Nde site to the 3′ end of Manduca EcR, which has an engineered HindIII site at the 3′ end as described above.

A fragment containing the herpes simplex VP16 transactivation domain was cloned from plasmid 35S/USP-VP16 (U.S. Pat. No. 5,880,333) using the PCR primers 5′-aagcttgcccccccgaccg-3′ (SEQ ID NO:11) (placing a HindIII site at the 5′ end of the domain) and 5′-tctagaggatcctacccaccgtact-3′ (SEQ ID NO:12) (placing an inframe stop codon followed by BamHI and Xbal sites at the 3′ end of the domain). The VP16 domain was fused in frame to the 3′ end of the E domain of the ecdysone receptor using the HindIII site 3′ to EcR clone and the HindIII site engineered at the 5′ end of VP16.

The plasmid pPacU (Courey A J and Tjian R (1988) Cell 55, 887-898) was used as the starting vector for expression constructs. The truncated Manduca EcR-VP16 was ligated into pPacU using the BamHI sites flanking the coding region to create the construct referred to as MMV.

Cell-Based Assay

An in vivo cell based assay was used to measure transcriptional activation by the EcR receptor plasmid in the presence of the chemical ligands as described above. S2 Drosophila cells (ATCC CRL-1963) (commonly known as cells from the fruit fly) were transiently transfected with luciferase reporter (pCGS154) and receptor expression plasmid (MMV) using the calcium phosphate precipitation procedure (Di Nocera and David (1983) PNAS 80, 7095-7098). S2 cells were plated in 96 well format at a density of 2×10⁵ in 166.6 μl of Schneider's Drosophila media supplemented with antibiotics and 10% heat inactivated fetal bovine serum (GIBO-BRL). The following day, 33.4 μl of a calcium phosphate precipitate containing 3-6 ng of pCGS154 reporter plasmid, and 3-6 ng of EcR receptor plasmid MMV along with salmon sperm DNA, to a total of 400 ng DNA per well were added. Chemical ligands (cyanoenamine test compounds) were added 16-24 hours after DNA addition to the cells at a final concentration of 2 μM. Cells were then harvested and extracted 24 hours after chemistry addition following the procedures for the luciferase assay by centrifuging and resuspending the cell pellets in 100 μl of cell culture lysis reagent (Promega). Luciferase activity was quantitatively determined using chemiluminescence (Promega) using an analytical luminescence model 2001 luminometer. Results were normalized as a ratio of induction relative to the reporter construct without chemical ligand addition.

The results are summarized in Table C below.

TABLE C

| | Gene switch activity |
|---|---|
| CHEMISTRY | fold induction |
| 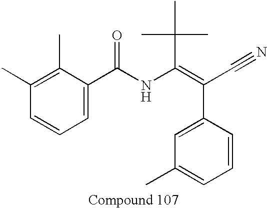 | 66 |
| 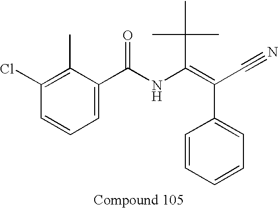 | 191 |
| 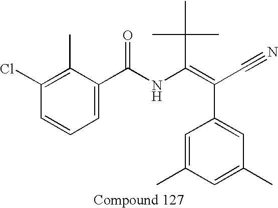 | 225 |
| 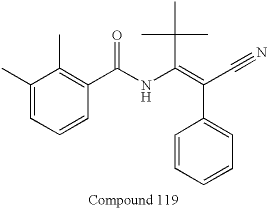 | 636 |
| 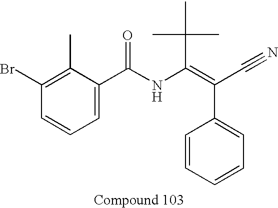 | 1112 |
| 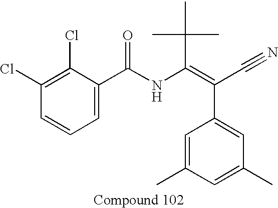 | 1145 |
| 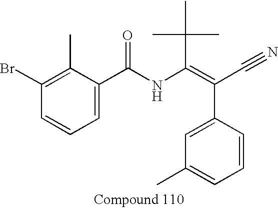 | 1012 |
| 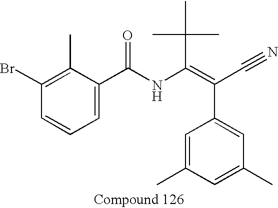 | 1121 |
| 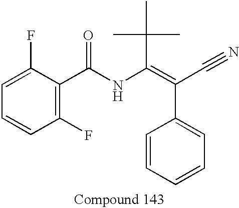 | 29 |
| 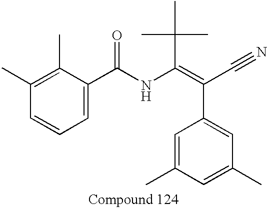 | 893 |
| 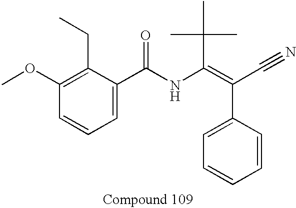 | 239 |
| 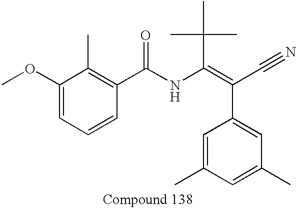 | 314 |
| 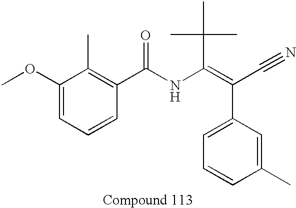 | 364 |
| 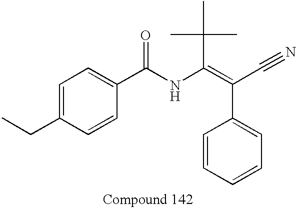 | 394 |
| 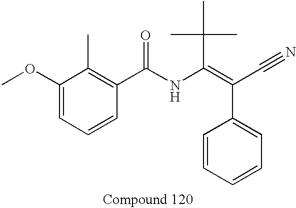 | 913 |

It will be understood that various details of the invention may be changed without departing from the scope of the invention. Furthermore, the foregoing description is for the purpose of illustration only, and not for the purpose of limitation—the invention being defined by the claims.

What is claimed is:

1. A compound comprising Formula I 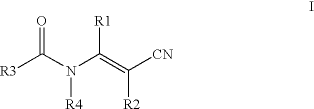
wherein:
R1
is a branched chain C3 to C8 alkyl, C3 to C8 cycloalkyl, C4 to C8 alkyl-substituted alkyl, bicycloalkyl, 1-adamantyl, polyhaloalkyl, trialkylsilyl, unsubstituted phenyl, or substituted phenyl;
R2 and R3
are each independently of the other unsubstituted or substituted aromatic rings, chosen from phenyl, pyridyl, pyrimidinyl, furyl, thiophenyl, pyrazinyl, pyrrolyl, pyrazolyl, 1,2,4-triazolyl, naphthyl, fluorenonyl, xanthenyl, 4-oxo-1,4-dihydro-(1,8)naphthyridinyl, thiazolyl, isothiazolyl, 1,3,4-thiadiazolyl, benzo-1,2,3-thiadiazolyl, oxazolyl, imidazolyl, quinolinyl, or isoquinolinyl, where a substituent on the rings is one or more chosen independently from hydrogen, C1 to C4 alkyl, alkoxy, alkoxyalkyl, hydroxy, amino, alkylamino, dialkylamino, acylamino, halo, haloalkyl, hydroxyalkyl, dihydroxyalkyl, alkoxycarbonyl, alkylaminocarbonyl, dialkylaminocarbonyl, unsubstituted or substituted alkylphenyl, unsubstituted or substituted phenyl, unsubstituted or substituted phenoxy, nitro, cyano, alkylthio, alkylsulfonyl, aminoalkyl, carboxyalkyl, or sulfonylalkyl; and
R4
is hydrogen, alkylthio, alkylthioalkyl, alkyloxyalkyl, acyloxyalkyl, alkyl, acyl, trialkylsilyl, or is taken together with R3 and the O in Formula I to form a lactone ring;
and the salts, stereoisomers, and tautomers thereof.

2. The compound of claim 1, wherein R1 is tert-butyl.

3. The compound of claim 1, wherein at least one of R2 and R3 is substituted with a substituent forming a cyclic structure on adjacent atoms of the aromatic ring.

4. The compound of claim 3, wherein the substituent is selected from the group consisting of 1,2-methylenedioxy and 1,2-difluoromethylenedioxy.

5. The compound of claim 1, wherein R2 is selected from the group consisting of phenyl, 3,5-dimethylphenyl, 2,4-dimethylphenyl, 3-methylphenyl, 4-methylphenyl, 2-methylphenyl, and 3,4-methoxydioxyphenyl.

6. The compound of claim 1, wherein R3 is selected from the group consisting of phenyl, 3-pyridyl, 3-methoxy-2-methylphenyl, 3-ethoxy-2-methylphenyl, 3-methoxy-2-ethylphenyl, 4-ethylphenyl, 2,6-difluorophenyl, 2,3-dimethylphenyl, 3-chloro-2-methylphenyl, and 3-bromo-2-methylphenyl.

7. The compound of claim 1, wherein halo is selected from the group consisting of fluoro, chloro, bromo, iodo, and combinations thereof.

8. The compound of claim 1, wherein Formula I is in its tautomeric form as Formula II:

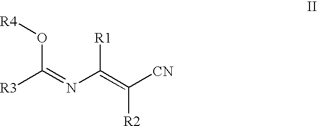

9. The tautomeric compound of claim 8, wherein R3 and R4 and O together form a cyclic structure resulting in a lactone.

10. The compound of claim 9, wherein the lactone is selected from the group consisting of:

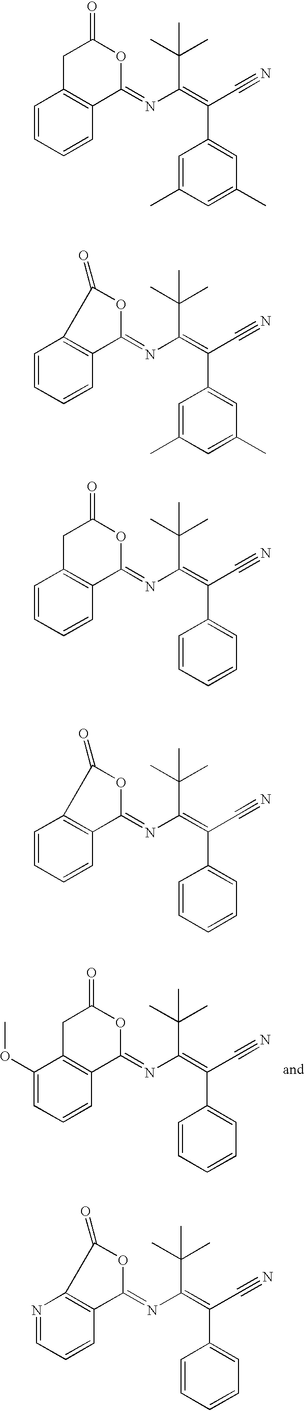

11. The compound of claim 1, wherein Formula I is in its isomeric form as Formula III:

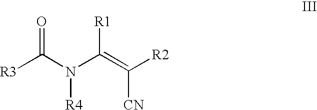

12. The isomeric compound of claim 11, wherein R1 is tert-butyl, R2 is 3,5-dimethylphenyl, and R3 is fluoromethylphenyl or 2-methyl-3-methoxyphenyl.

13. The isomeric compound of claim 12, wherein the compound is selected from the group consisting of:

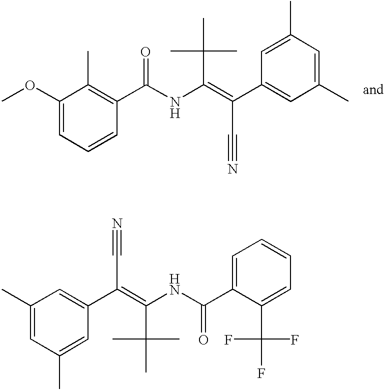

14. The compound of claim 1, wherein the compound is selected from the group consisting of:

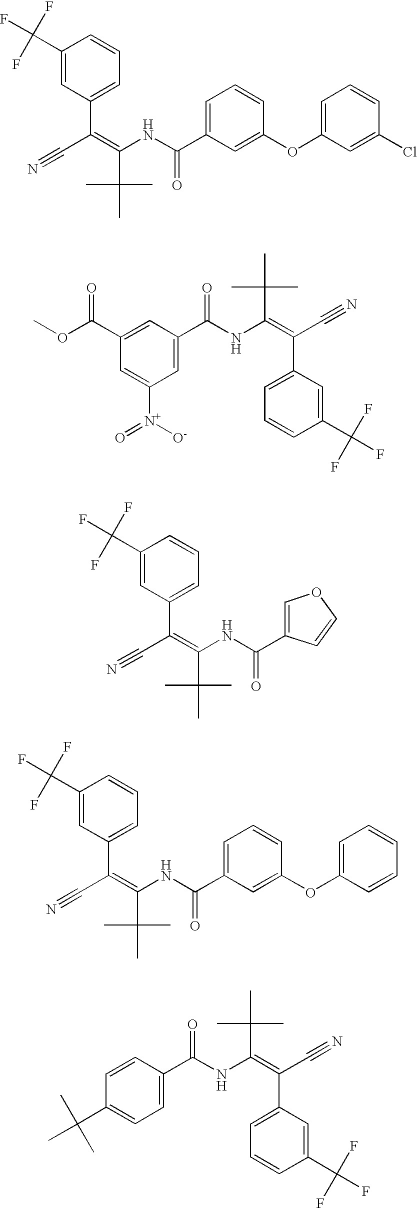

15. A method of controlling gene expression comprising contacting an ecdysone receptor gene switch with a compound of Formula I 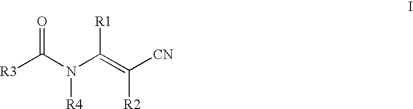
wherein:
R1
is a branched chain C3 to C8 alkyl, C3 to C8 cycloalkyl, C4 to C8 alkyl-substituted alkyl, bicycloalkyl, 1-adamantyl, polyhaloalkyl, trialkylsilyl, unsubstituted phenyl, or substituted phenyl;
R2 and R3
are each independently of the other unsubstituted or substituted aromatic rings, chosen from phenyl, pyridyl, pyrimidinyl, furyl, thiophenyl, pyrazinyl, pyrrolyl, pyrazolyl, 1,2,4-triazolyl, naphthyl, fluorenonyl, xanthenyl, 4-oxo-1,4-dihydro-(1,8)naphthyridinyl, thiazolyl, isothiazolyl, 1,3,4-thiadiazolyl, benzo-1,2,3-thiadiazolyl, oxazolyl, imidazolyl, quinolinyl, or isoquinolinyl, where a substituent on the rings is one or more chosen independently from hydrogen, C1 to C4 alkyl, alkoxy, alkoxyalkyl, hydroxy, amino, alkylamino, dialkylamino, acylamino, halo, haloalkyl, hydroxyalkyl, dihydroxyalkyl, alkoxycarbonyl, alkylaminocarbonyl, dialkylaminocarbonyl, unsubstituted or substituted alkylphenyl, unsubstituted or substituted phenyl, unsubstituted or substituted phenoxy, nitro, cyano, alkylthio, alkylsulfonyl, aminoalkyl, carboxyalkyl, or sulfonylalkyl; and
R4
is hydrogen, alkylthio, alkylthioalkyl, alkyloxyalkyl, acyloxyalkyl, alkyl, acyl, trialkylsilyl, or is taken together with R3 and the O in Formula I to form a lactone ring;
and the salts, stereoisomers, and tautomers thereof.

16. The method of claim 15, wherein R1 is tert-butyl.

17. The method of claim 15, wherein at least one of R2 and R3 is substituted with a substituent forming a cyclic structure on adjacent atoms of the aromatic ring.

18. The method of claim 17, wherein the substituent is selected from the group consisting of 1,2-methylenedioxy and 1,2-difluoromethylenedioxy.

19. The method of claim 15, wherein R2 is selected from the group consisting of phenyl, 3,5-dimethylphenyl, 2,4-dimethylphenyl, 3-methylphenyl, 4-methylphenyl, 2-methylphenyl, and 3,4-methoxydioxyphenyl.

20. The method of claim 15, wherein R3 is selected from the group consisting of phenyl, 3-pyridyl, 3-methoxy-2-methylphenyl, 3-ethoxy-2-methylphenyl, 3-methoxy-2-ethylphenyl, 4-ethylphenyl, 2,6-difluorophenyl, 2,3-dimethylphenyl, 3-chloro-2-methylphenyl, and 3-bromo-2-methylphenyl.

21. The method of claim 15, wherein halo is selected from the group consisting of fluoro, chloro, bromo, iodo, and combinations thereof.

22. The method of claim 15, wherein Formula I is in its tautomeric form as Formula II:

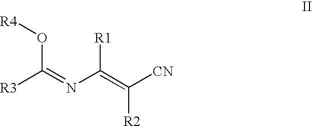

23. The method of claim 22, wherein in the tautomeric form, R3 and R4 and O together form a cyclic structure resulting in a lactone.

24. The method of claim 23, wherein the lactone is selected from the group consisting of:

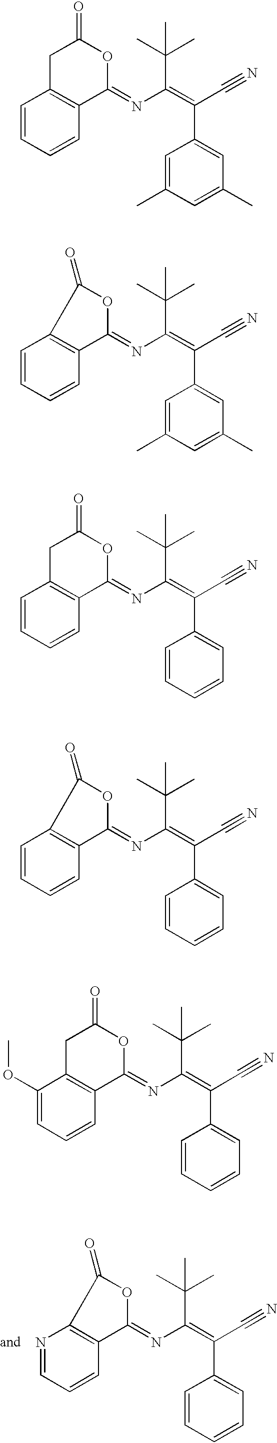

25. The method of claim 15, wherein Formula I is in its isomeric form as Formula III:

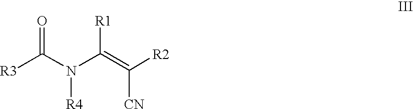

26. The isomeric method of claim 25, wherein R1 is tert-butyl, R2 is 3,5-dimethylphenyl, and R3 is 2-trifluoromethylphenyl or 2-methyl-3-methoxyphenyl.

27. The isomeric method of claim 26, wherein the compound is selected from the group consisting of:

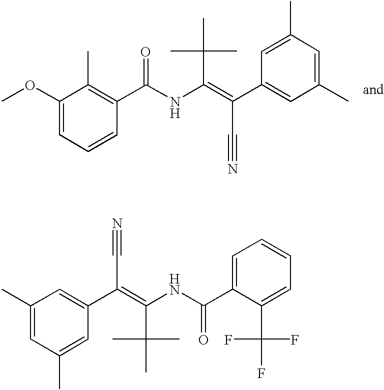

28. The method of claim 15, wherein the compound is selected from the group consisting of: [chemical structure]
